(19) United States
(12) Patent Application Publication
(10) Pub. No.: US 2025/0186708 A1
(43) Pub. Date: Jun. 12, 2025

(54) GAS TRAP DEVICE

(71) Applicant: TESSEN SOLUTIONS LIMITED, Cork (IE)

(72) Inventors: Vincent FORDE, Cork (IE); Pauric CAREY, Cork (IE); James ROCHE, Cork (IE)

(73) Assignee: TESSEN SOLUTIONS LIMITED, Cork (IE)

(21) Appl. No.: 18/835,193

(22) PCT Filed: Jan. 27, 2023
(86) PCT No.: PCT/EP2023/052032

§ 371 Date: Aug. 1, 2024

(30) Foreign Application Priority Data
Feb. 2, 2022 (EP) 22154850.6

(51) Int. Cl. A61M 5/36; A61M 39/20

(52) U.S. Cl. CPC A61M 5/36; A61M 39/20; A61M 2039/205

(57) ABSTRACT

Gas trap devices and methods of use such as uses for intravenous medial use are described. They effectively prevent gas bubbles from migrating distally towards a patient in use. They may have a priming port which is used only once and permanently sealed at priming. There may be a particle filter to also prevent migration of particles towards the outlet, and this may be across the full cross section of the chamber between the diverter and the outlet conduit.

### INTRODUCTION

The present invention relates to a gas trap for uses such as infusion apparatus for human or animal use.

When administering hazardous drugs, biohazardous fluids or other fluids it is important to maintain a closed system that eliminates ingress of contaminants and egress of toxic liquids, gases or vapours. In practice, a closed system is preferred end-to-end to increase safety for both the patient and the healthcare practitioner. A closed system infers that the drug is delivered directly into the patient's venous system without opening the IV system that carries the fluids from the reservoir, typically a bag or bottle, through a tube system and venous entry device, such as a catheter or needle. In practice there may be one or several devices attached to the line, for example, to allow access where additional drugs are required. There are a variety of components that enable access to the IV system, such as stopcocks, multi-way taps, y-connectors, Luer connectors or other IV components intended to be accessed by a third-party device. Each time a break is made in the line, or a component attached or accessed, there is an increased risk of pressure drop that can result in air being pulled into the IV line. For example, a standard Luer or push connection may be erroneously left open or under-tightened so as to not create an air-tight seal. It is therefore a recognised preference among healthcare practitioners to reduce or eliminate the number of connection devices or manual manipulations of an IV. Furthermore, the liquid drugs being delivered may be volatile or chemically unstable, resulting in degassing of the liquids.

Another source of air in IV lines comes from temperature changes in the liquid as they are administered over a period of time. Many fluids, such as blood and other drugs, are stored in refrigerators as best practice, these drugs are then left to warm to ambient temperatures or actively warmed to body temperature to reduce hypothermic reactions in patients. For chemotherapy or other hazardous drugs, compounding activities are typically done in the Pharmacy in hospitals, under controlled conditions that reduce risk of exposure of the drug vapours or spills of the liquid, which may be hazardous to human health. Exposure of hazardous drugs is a recognised hazard that results in increased rates of infertility, reproductive issues and cancers among healthcare workers.

When administering chemotherapy or other hazardous drugs, nurses will employ a variety of methods to set up the IV system. The drug may be given by gravity induced flow or by IV pump. The drug may be given as a single dose or combined with other fluids, a setup typically known as piggyback or secondary infusion. The setup of the giving set can vary depending on drugs being used, hospital policy, economic capacity of the facility or any other reasons. In any IV infusion, a closed system is preferred to protect both healthcare worker and patient.

For chemotherapy administration, a typical scenario is for the drug to be compounded in the Pharmacy and delivered to the oncology department, where administration is handled by nurse professionals. These nurses will often wear appropriate PPE (Personal Protective Equipment) and employ a sterile process while hanging the IV fluids. In some scenarios a patient may be receiving several fluids in a single chemotherapy sitting and they may already have an active infusion underway. Typical chemotherapy sittings can range from one to more than one drug, as well as non-hazardous drugs such as saline solution, being administered for up to or more than one hour at a time.

Air-in-Line is the commonly used term to describe bubbles of air that are present in the IV tube during administration, these bubbles being recognised as a hazard due to the risk of air embolism. Vascular Air Embolism (VAE) is the result of any quantity of air entering the venous system. Air is foreign to the venous system and can trigger a number of adverse reactions, such as inflammation, destruction of endothelial cells, inducement of the coagulation cascade, blockage of blood flow to critical organs or tissue, heart complications. The most serious events occur when air enters the arterial blood supply, which can result in migration of micro-bubbles into cortical tissue, resulting in ischemic strokes and cognitive dysfunction in patients. Patients with a patent foramen ovalae, commonly known as hole in the heart, a condition which is believed to affect up to 40% of the adult population and all infants, are at much higher risk of air migrating into the left side of the heart and passing freely to the brain. This is known as a paradoxical air embolism and can result in catastrophic damage to brain function with even tiny amounts of air. For these reasons, a high degree of safety is practiced in healthcare to ensure air is not allowed to pass into the patient's vasculature.

For many critical drugs that have a short half-life, such as chemotherapy, or where accuracy of flow rate is important, active devices such as IV pumps are used to control flow. These pumps may be activated by syringe or peristaltic of other method to pull a specified volume of the fluids form the reservoir to the patient. The IV pumps usually include a sensor device to identify when Air-in-line is present and a warning system to alert medical professionals to the danger, the pump then stopping the IV infusion until the air is cleared. Typically, a nurse must manually intervene to remove the bubble, often using a variety of methods including tapping, flicking or agitating the line, or attaching a third-party device to suck the air from the line. The method of removing the AIL can depend on the IV setup.

Various devices are disclosed in our co-pending specification number WO2020/156666. Such devices include a diverter between chamber inlet and outlet ports, and a diffuser in the inlet with holes in a radial arrangement.

During intravenous (IV) infusion natural degassing can occur. In one example, this can occur if there are two or more chemically incompatible fluids, leading to bubbles of gas that can pose a threat to the health of a patient. Moreover, precipitate forming from fluids may also cause harm.

The present invention addresses the above issues.

### SUMMARY OF THE INVENTION

We describe a gas trap device for medical supply of liquids to a human or animal body, the device comprising:
a chamber,
an inlet port,
a diffuser in the inlet port, the diffuser comprising openings for radial flow into the chamber of fluid relative to an inlet port axis,
an outlet port spaced apart distally from the inlet port and being linked with a conduit within the chamber and said conduit having an opening within the chamber, and
a flow diverter mounted distally of the inlet port and proximally of said conduit.

In preferred examples, the diverter is configured for flow of inlet fluid to change direction through at least 80° from an inlet port axis and then to flow radially through diffuser openings having an area of less than half of an area around said axis. In preferred examples, the diffuser openings have an area which is in the range of 20% to 40% of an area around the inlet axis.

In preferred examples, there are in the range of two to four diffuser openings. In preferred examples, the diverter is located within the chamber in a region less than 40% from the proximal end of the chamber, and preferably less than 25% from the proximal end of the chamber.

In preferred examples, the diverter forms an end surface of the inlet port, forcing inlet fluid to flow through the diffuser openings. In preferred examples, the diverter has a proximally facing surface which is tapered distally and radially on a radially outer side of the openings, and this taper is optionally in the range of 5° to 20° relative to a place normal to an inlet port axis.

In preferred examples, the diverter comprises a projection facing proximally towards the inlet port, preferably into the inlet port. In preferred examples, the diverter comprises an annular surface around said projection, said surface being concave. In preferred examples, said annular surface terminates at said inlet port openings and walls between said openings.

In preferred examples, the diverter distal surface is convex. In preferred examples, the chamber is substantially spherical. In preferred examples, the chamber is of rigid material, not being compressible by hand. In preferred examples, the chamber has a rib or ridge extending around its circumference. In preferred examples, said rim is formed by two chamber parts being sealed together.

In preferred examples, the device further comprises a single use priming port, configured to allow venting of the chamber during priming and to be permanently sealed upon completion of priming. In preferred examples, the priming port comprises a body forming a vent opening and a cap which may be inserted but not manually removed.

In preferred examples, the cap and the vent opening have inter-engaging features which are engageable by pushing the cap into the opening. In preferred examples, the cap and the opening comprise inter-engaging ridge and groove. In preferred examples, the cap has a curved exposed surface without a hand grip. In preferred examples, the cap is configured to be normally activated by hand.

In preferred examples, the cap is linked with the vent opening by a ribbon. In preferred examples, the ribbon has a ring engaged around the vent opening to allow rotation about an axis of the vent opening. In preferred examples, the cap is configured to close only without removal. In preferred examples, the cap and the vent opening have inter-engaging features which provide a snap-fit lock with pushing of the cap into the vent opening. In preferred examples, the chamber comprises a transparent section. In preferred examples, the chamber comprises a rough surface finish to limit visibility into the chamber.

In preferred examples, the chamber has a viewing window that shows a proximal volume of the chamber, and optionally the remaining portion of surface area is treated to reduce or limit visibility such that a nurse is encouraged to orient the device into a preferred position to observe the internal chamber volume which is uppermost in use and to aid visibility of any air during priming or functional performance.

In preferred examples, a proximal transparent section of the chamber wall defines less than 45% of the total internal chamber volume, and the remaining chamber wall is obscured relative to the transparent section. In preferred examples, the remaining chamber wall surface is roughened to obscure certain regions.

In preferred examples, an indicator line is formed as a negative or positive mark or indent on the chamber wall outer surface by moulding, etching, painting or transferring a mark by any other means, the purpose of this mark or indent being to show safe use levels of internal fluids to inform the clinician of the volume of air in the device. In preferred examples, the indicator line is configured to be used to indicate a transition from fully or substantially transparent material to frosted or otherwise obscured chamber wall material.

In preferred examples, a particle filter is mounted within the chamber to block flow of particles towards the outlet. In preferred examples, the particle filter is mounted distally of the diverter and proximally of the outlet conduit.

In preferred examples, the particle filter is closer to the diverter than it is to the opening of the outlet conduit. In preferred examples, the particle filter extends across a full cross section of the chamber. In preferred examples, the particle filter comprises a mesh mounted across a frame and the frame comprises a rim engaging an internal surface of the chamber.

In preferred examples, the frame comprises a rim extending proximally from a plane of the filter, and the rim is optionally circular in shape. In preferred examples, the particle filter comprises an outer region configured to attract particles.

In preferred examples, said region is charged. In preferred examples, said outer region is annular.

In preferred examples, the particle filter comprises a filter element with a tubular configuration surrounding an axis of the outlet conduit. In preferred examples, the particle filter comprises a filter element substantially surrounding a space between the outlet opening and the diverter.

In preferred examples, the particle filter comprises a filter element which forms said outlet conduit.

In preferred examples, the particle filter comprises a filter element which forms a closed volume such as being spherical around the outlet opening.

In preferred examples, the device comprises a priming port and said priming port comprises a backcheck valve arranged to allow outflow of gas and to prevent inflow of gas. In preferred examples, the backcheck valve comprises a plurality of converging walls arranged to deflect towards each other and to remain close together with inflow, and to separate to provide an opening with outflow.

In preferred examples, the device comprises a priming port, and said priming port comprises a filter to restrict flow of liquid out of the priming port but to allow flow of gas out of the priming port. In preferred examples, the priming port filter comprises a mesh with a hydrophobic material to help prevent outflow of liquid during priming.

We also describe a flow line assembly for delivery of a medicinal liquid, the assembly comprising a flow line, a gas trap device of any example described herein, said inlet and said outlet being sealed to ends of the flow line.

In preferred examples, the device comprises a collar for receiving and bonding with a flow line at either or both ends, or a tube configured to insert into the line and bonding thereto.

In preferred examples, an assembly comprises a flow line, a gas trap device of any example, and a particle filter discretely located in said line separately from the gas trap device. In preferred examples, the particle filter is downstream of the gas trap device.

We also describe a flow line assembly comprising a device of any example, a secondary fluid reservoir linked to the device inlet port, a primary reservoir linked with a connector by a primary line and said connector is also connected to the device outlet port and to a patient interface, and a clamp distally of the connector.

We also describe a method of use of a gas trap device comprising a chamber, an inlet port, an outlet port spaced apart distally from the inlet port, a flow diverter within the chamber distally end of the inlet port, and a priming port, the method comprising the steps of allowing flow of a priming fluid into the chamber to cause the chamber to fill with said priming fluid and expel gas from the chamber, via the priming port, closing the priming port after expelling the gas, and then allowing a fluid to flow through the device. In preferred examples, the priming fluid is saline.

In preferred examples, either the priming port or the outlet port is closed and the priming fluid is allowed to flow through the inlet port until it fills the chamber, upon which the priming port is sealed and flow is prevented through the inlet until the outlet is opened.

In preferred examples, the inlet port is closed, the outlet port is connected to a connector for a primary line, a line distally of the connector is closed, a flow is allowed through the primary line so that it is diverted via the connector back upwardly into the device until it fills the device chamber, the priming port is sealed, the line distally of the connector is opened, and the inlet port is connected to a secondary line. In preferred examples, entrained air from the device attached to an IV line is expelled during priming.

In preferred examples, the device is used with a secondary or piggyback IV setup, with the device being attached to a connector on an IV line, and a connector on the distal end of the device being connecting to a female connector on the IV system. In preferred examples, the primary line has a component to stop flow in the secondary line from travelling into the primary line in the wrong direction, towards the reservoir.

In preferred examples, the device is connected at a distal end of the secondary line which connects to the Y-connector of the primary line to capture air from the secondary infusion line, and when air is in the IV system it can be routed through the primary or secondary lines by forward or reverse flow direction, and will be captured in a closed air trap device, and when the secondary line is primed drugs can be alternately selected by the medical professionals as required.

In preferred examples, for vented back-priming, the device is placed on a Y-connector so that fluids can be directed to flow into the device, which is connected to the Y-connector and the secondary line which is further attached to the proximal connector on the device, and optionally the venting port cap can be open for air to escape to atmosphere, or closed.

In preferred examples, for closed back-priming flow is reversed using primary fluids to fill the chamber, the air in the chamber is expelled as the chamber fills with fluids, which flows up through the distal port end and into the chamber through an intake end or opening of the outflow tube/conduit and as the chamber is filled with fluids the air is expelled and the fluids continue to travel up through holes into the secondary line that is connected via a connection through the outlet port opening, and the venting cap is closed to prevent air from escaping to atmosphere.

In preferred examples, once the secondary IV line is primed, flow is reversed to flow in the ‘normal’ direction, from reservoir to patient. In preferred examples, the chamber is sealed as a first step in a back-priming process by closing the vent, to ensure a closed system while priming is taking place and so that air present in the device chamber does not need to vent through an open port and can be expelled fully from the chamber through holes into the secondary line and secondary reservoir or vented to atmosphere.

In preferred examples, the device is positioned distal to a Y-connector as a component of the IV line, such that it can capture air from fluids of any line attached proximally to the device. In preferred examples, the device is positioned proximal to an IV pump as a component of an IV line, such that it can capture air from fluids of any line attached proximally to the device. In preferred examples, the device is positioned at the distal end of an IV line.

In preferred examples, the device is positioned at any location on any fluid tube, such that fluids can flow into the device from any direction, forward or backward, and the device can be vented or can capture air from fluids that flow into the device.

We also describe a gas trap device for supply of liquids such as medical supply of liquids to a human or animal body, the device comprising a chamber, an inlet port, an outlet port spaced apart distally from the inlet port, and a flow diverter at the distal end of the inlet port.

In one example, the diverter is configured to cause flow of inlet fluid to change direction through at least 80° and preferably at least 90° from an inlet port axis, and then to flow radially through openings having an area of less than half of an area around said axis.

In one example, the openings have an area which is in the range of 20% to 40% of an area around the inlet port axis. In one example, there are in the range of two to four openings, and in one preferred example there are three openings. In one example, the diverter comprises a projection facing proximally towards the inlet port, preferably into the inlet port. In one example, the diverter comprises an annular surface around said projection, said surface being concave. In one example, said annular surface terminates at its outer periphery with said openings and walls between said openings.

In one example, the diverter has a proximally facing surface which is tapered distally and radially on a radially outer side of the openings. In one example, the diverter distal surface is closer to the proximal end of the chamber than to the distal end of the chamber. In one example, the diverter distal surface is convex. In one example, the chamber is substantially spherical. In one example, the chamber is of rigid material, not being compressible by hand. In one example, the chamber has a rib or ridge extending around its circumference. In one example, said rim is formed by two chamber parts being sealed together.

In one example, the device further comprises a single use priming port, configured to allow venting of the chamber during priming and to be permanently sealed upon completion of priming. In one example, the port comprises a body forming a vent opening and a cap which may be inserted but not manually removed. In one example, the cap and the vent opening have inter-engaging features which are engageable by pushing the cap into the opening.

In one example, the cap and the opening comprise a ridge and an inter-engaging groove. In one example, the cap has a curved exposed surface without a hand grip. In one example, the cap is configured to be normally activated by hand. In one example, the cap is linked with the vent opening by a ribbon. In one example, the ribbon has a ring engaged around the vent opening to allow rotation about an axis of the vent opening. In one example, the cap is configured for closure only without removal.

In one example, the cap and the vent opening have inter-engaging features which provide a snap-fit lock with pushing of the cap into the vent opening.

In one example, the chamber comprises a transparent section. In one example, the chamber comprises a rough surface finish to limit view into the device. In one example, the chamber has a viewing window that shows a preferred uppermost section of the internals, and optionally the remaining portion of surface area is treated to reduce or limit visibility such that the nurse is encouraged to orient the device into a preferred position to observe the internal and to aid visibility of any air during priming or functional performance.

In one example, an uppermost section of the chamber has less than 45% of the total internal liquid volume, is transparent and the remaining chamber wall below this is semi-transparent, with visibility still possible, but that portion of the device surface obscured.

In one example, the chamber surface is roughened to obscure certain regions. In some embodiments, an indicator line is formed as a negative or positive indent on the chamber surface during or after production by moulding, etching, painting or transferring a mark by any other means, the purpose of this mark or indent being to show the safe use levels of the internal fluids to inform the clinician of the volume of air in the device.

In one example, the mark or indent line is configured to be used to indicate a transition from fully or substantially transparent material to frosted or otherwise obscured material, We also describe a method of use of a gas trap device of example described herein, the method comprising the steps of allowing flow of a priming liquid into the chamber to cause the chamber to fill with said priming liquid and expel gas from the chamber, closing the priming port after expelling the gas, and then allowing a fluid to flow through the device.

In one example, the priming liquid is saline. In one example, for priming, either the vent port or the outlet port is closed, and the priming liquid is allowed to flow through the inlet port until it fills the chamber, upon which the priming port is sealed, and flow is prevented through the inlet until the outlet is opened.

In one example, for use with a secondary line the inlet port is closed, the outlet port is connected to connector for a primary line, a line distally of the connector is closed, a flow is allowed through the primary line so that it is diverted via the connector back upwardly into the chamber until it fills the chamber, the priming port is sealed, the line distally of the connector is opened, and the inlet port is connected to the secondary line.

In one example, normally entrained air from the device attached to an IV line is expelled during priming.

In one example, the device is used with a secondary or piggyback IV setup, with the device being attached to a connector on the IV line by a Y-connector or Luer connector or any other type of connector, the male connector on the distal end of the device connecting to a female connector on the IV system.

In one example, two or more lines are connected together to deliver one or more drugs to a patient, using gravity or an IV pump to generate flow, in an arrangement for IV pump setup where the primary line has a Y-connector to connect the secondary line.

In one example, the primary line delivers a saline solution from a reservoir bag into a patient, and optionally an IV pump is used, and the primary line is engaged by the pump to control flow rate of the IV fluids.

In one example, the primary line has components attached to it to facilitate flow, such as a pinch clamp to stop flow or a back-check valve to stop fluids from the secondary line from travelling into the primary line in the wrong direction, towards the chamber. In one example, the secondary line is used to administer drugs of a non-hazardous or hazardous nature, these drugs being medicinal for the patient, and optionally the Y-connector has a port to which the secondary line is attached.

In one example, the device is connected at the distal end of the secondary line which connects to the Y-connector of the primary line to capture air from the secondary line, and when the secondary line is primed drugs can be alternately selected by the medical professionals as required.

In one example, the device outlet port is connected to a Y-connector so that fluids can be directed to flow into the device via the Y-connector and the outlet port. A secondary line may be further attached to the inlet port, and optionally the venting port cap can be open while back-priming through the device and the air in the chamber is expelled through the priming port as the chamber fills with liquid which flow up through the distal outlet port end and into the chamber whereby the flow is reversed using primary liquids to fill the chamber and as the chamber is filled the air is expelled and the liquid continues to travel up through holes and escape to atmosphere or into the secondary line that is connected via a connection through the outlet port opening, and the venting cap is closed to seal the chamber.

In one example, once the secondary IV line is primed, flow is reversed to flow in the ‘normal’ direction, from reservoir to patient. In one example, the chamber is sealed as a first step in a back-priming process by closing the priming port, to ensure a closed system while priming is taking place and so that air present in the device chamber does not need to vent through an open port and can be expelled fully from the chamber through holes into the secondary line and secondary reservoir or vented to atmosphere.

In one example, the device is positioned distal to a Y-connector as a component of an IV line, such that it can capture air from fluids of any line attached proximally to the device. In one example, the device is positioned proximal to an IV pump as a component of the IV line, such that it can capture air from fluids of any line attached proximally to the device. In one example, the device is positioned at the distal end of an IV line.

In one example, the device is positioned at any location on any fluid tube, such that fluids can flow into the device from any direction and the device can be vented or can capture air from fluids that flow into the device.

In another embodiment the internal surface of the chamber of the device is coated with a surface treatment that has properties such as chemical or medicinal properties to enhance the function of the device. Examples of advantageous uses of such a device are in the fields of drug administration or biologics. For example, the coating could be a Heparin or other agent that acts against clotting of blood, or the coating could be an agent formulation that reduces propagation of bacteria on the surface of the device or within the fluids that pass through the device.

The coating or surface treatment may be applied to one or more parts on the internal or external or both surfaces of the device, and it may be applied to components or features of the device selectively, for example the diverter, extending from the inlet tube to the outlet tube.

In some embodiments, the surface treatment is applied by gas, vapour, bonding, coating, pasting or otherwise treating the surface with a chemical, water-based or oil-based solution. In another embodiment the surface treatment is applied in the manufacturing phase.

In some embodiments, the surface treatment is applied to individual components, select surfaces or entire surfaces prior to assembly. In another embodiment the surface treatment is applied to components, select surfaces or entire surfaces after assembly.

In a preferred embodiment the surface treatment is a chemical anticoagulant drug, such as Heparin, used to reduce clotting in blood that flows into the chamber and through the device. In a preferred embodiment the surface treatment is an anti-bacterial chemical agent, such as Hypochlorous Acid, Hydrogen Peroxide or other agent, used to reduce or eliminate the number of bacteria that can form on the surfaces of the material or in the fluids that flow into the chamber and through the device.

### DETAILED DESCRIPTION OF THE INVENTION

The invention will be more clearly understood from the following description of some embodiments thereof, given by way of example only with reference to the accompanying drawings in which:

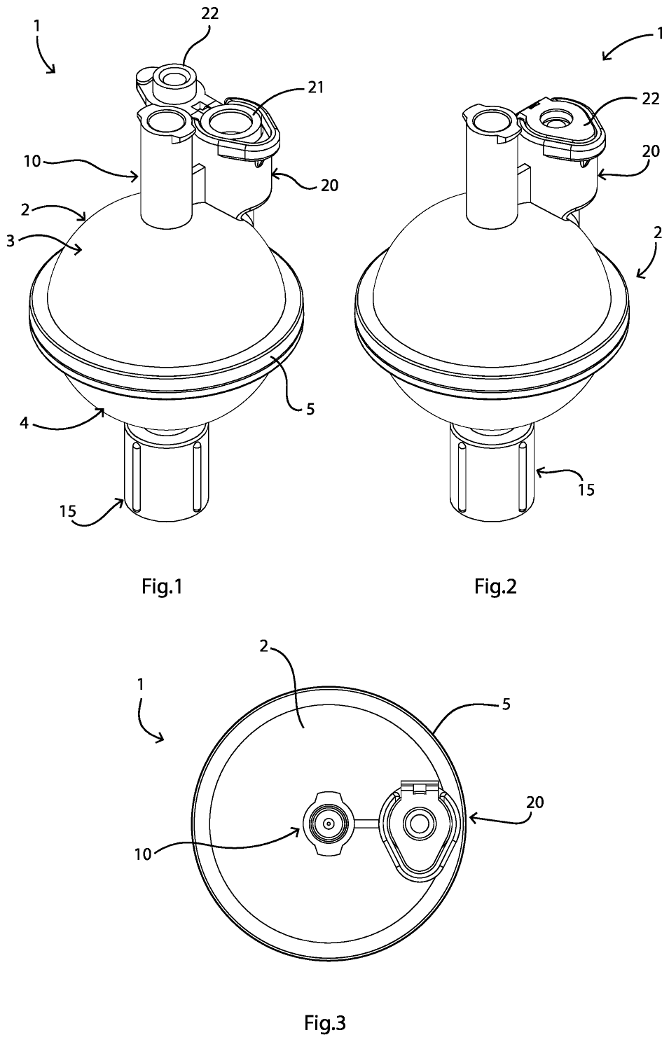

FIG. 1 is a perspective view of a gas trap device (“trap”) of the invention with a priming port open, and FIG. 2 is a similar view with the priming port closed, and FIG. 3 is a plan view of the trap with the priming port closed;

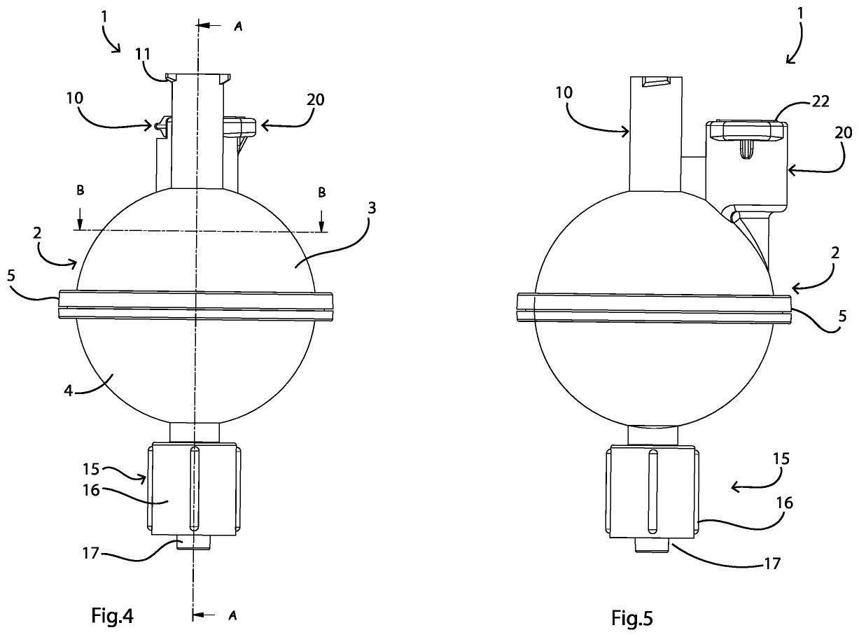

FIG. 4 is a front view of the trap, showing sections lines A-A and B-B, and FIG. 5 is a front view from another direction in which the priming port is not hidden;

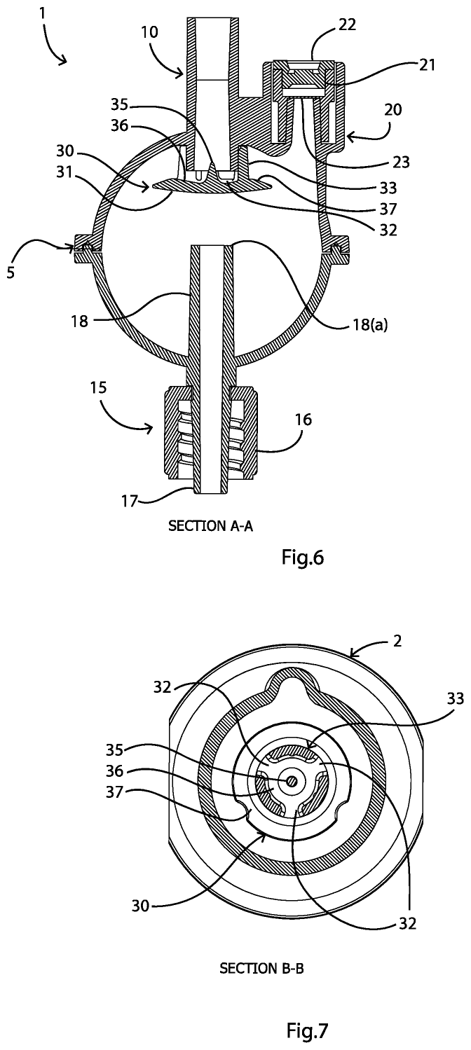

FIG. 6 is a sectional view in the direction of the arrows A-A of FIG. 4;

FIG. 7 is a cross-sectional view in the direction of the arrows B-B of FIG. 4;

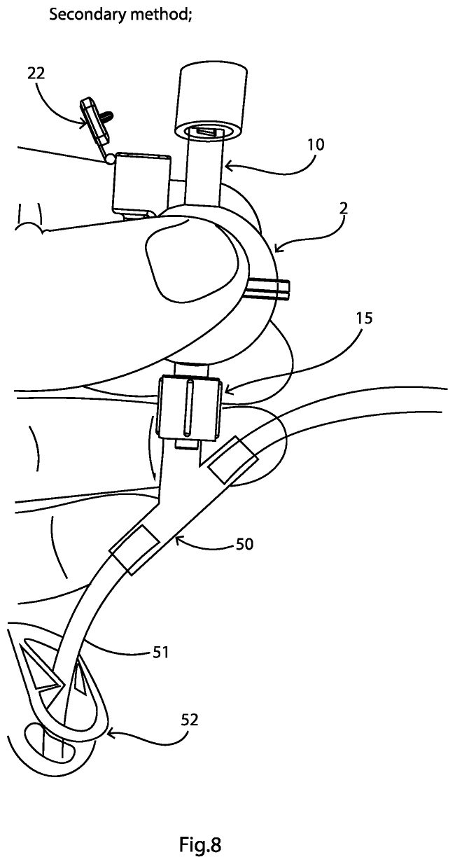

FIG. 8 is a diagram showing the trap device in use with a primary and secondary liquid supply mode;

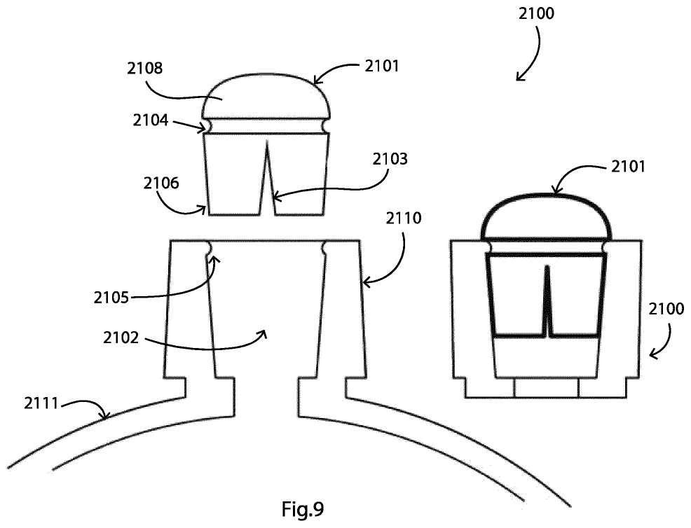

FIG. 9 is a cross sectional diagram showing another priming port and its sealing arrangement;

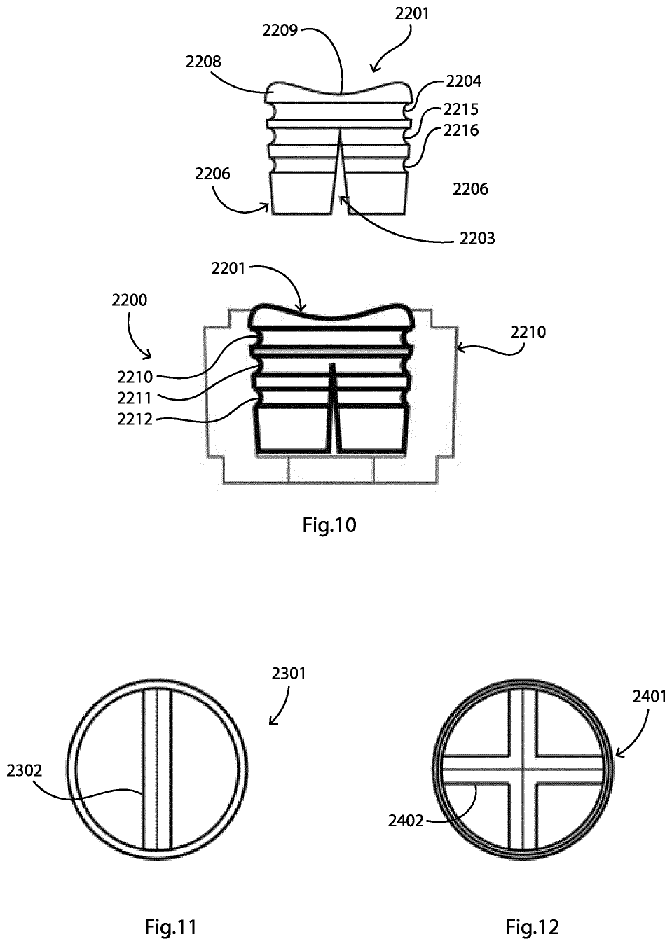

FIG. 10 shows an alternative priming port cap with a more positive sealing engagement; and FIGS. 11 and 12 are plan views of further alternative port caps, in this case with notches for engagement by a tool;

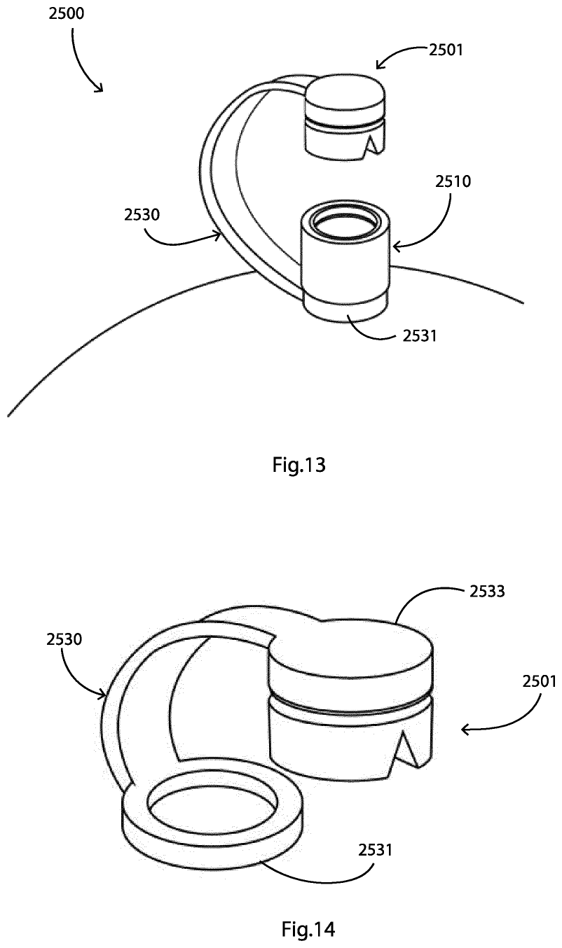

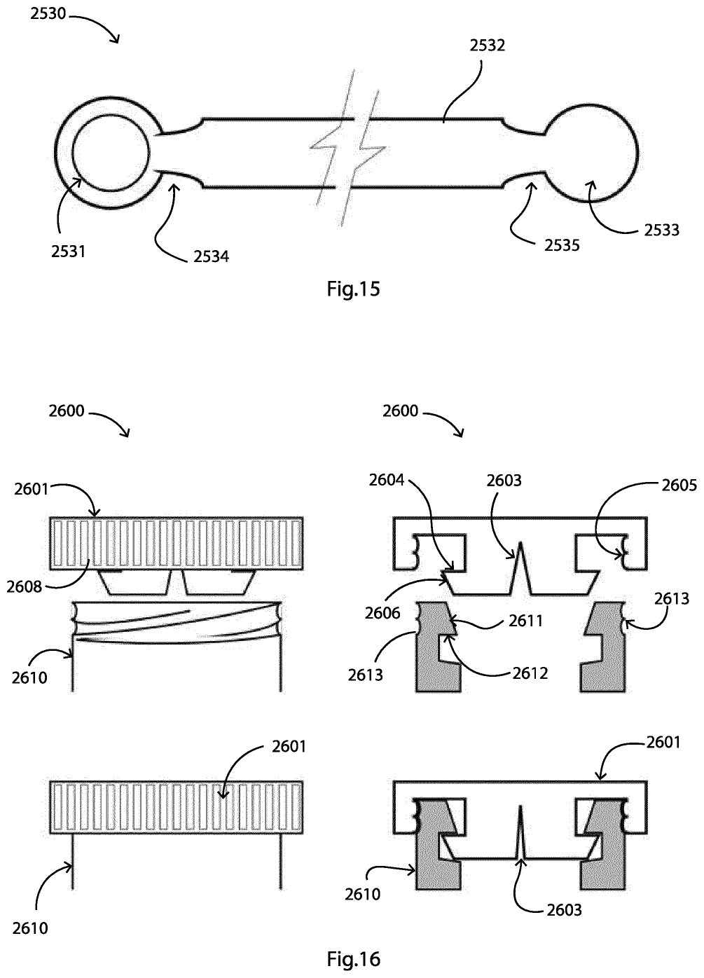

FIGS. 13 and 14 are perspective views showing an alternative priming port, in this case with the cap being linked by a ribbon, and FIG. 15 is a plan view of priming port components;

FIG. 16 is a set of side and sectional views of an alternative priming port, in this case with Luer threads for closing a cap;

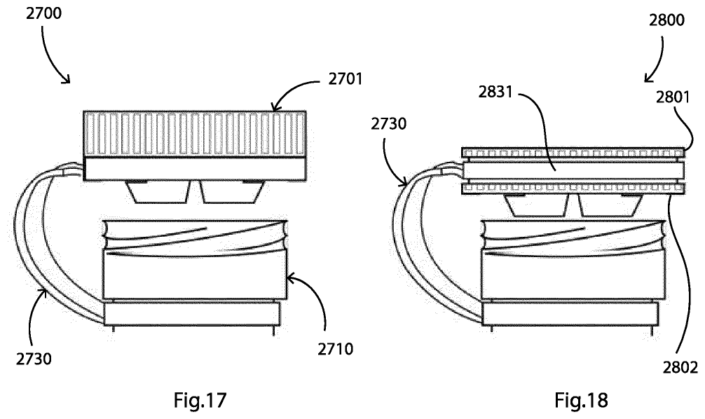

FIGS. 17 and 18 are side views of alternative priming ports, in this case with threads for closing the caps and with connector ribbons;

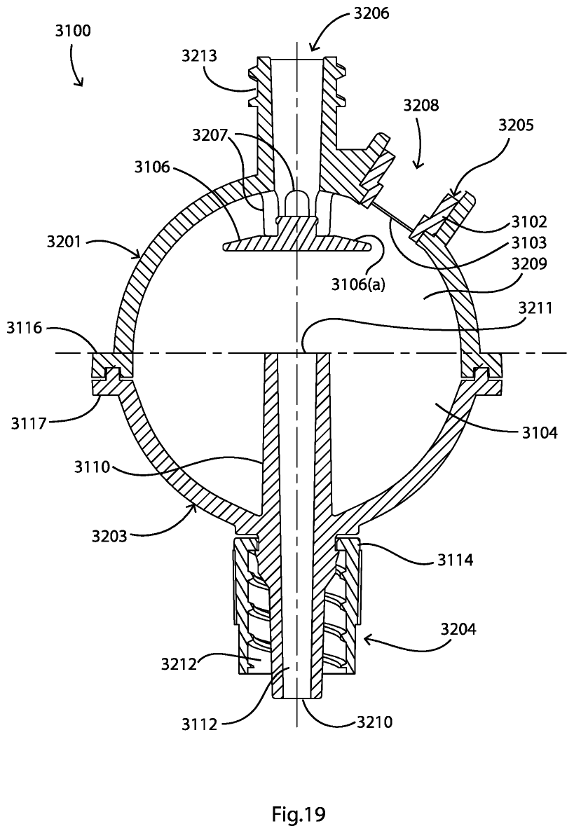

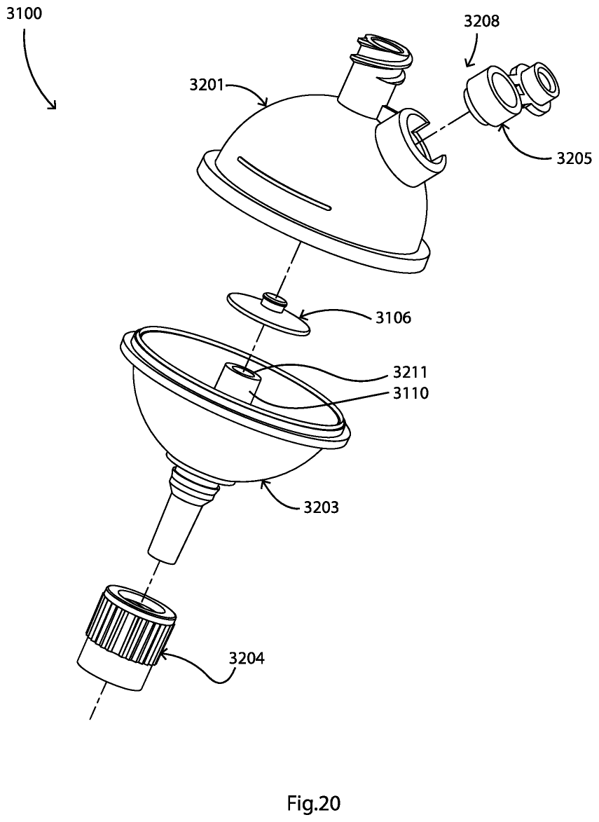

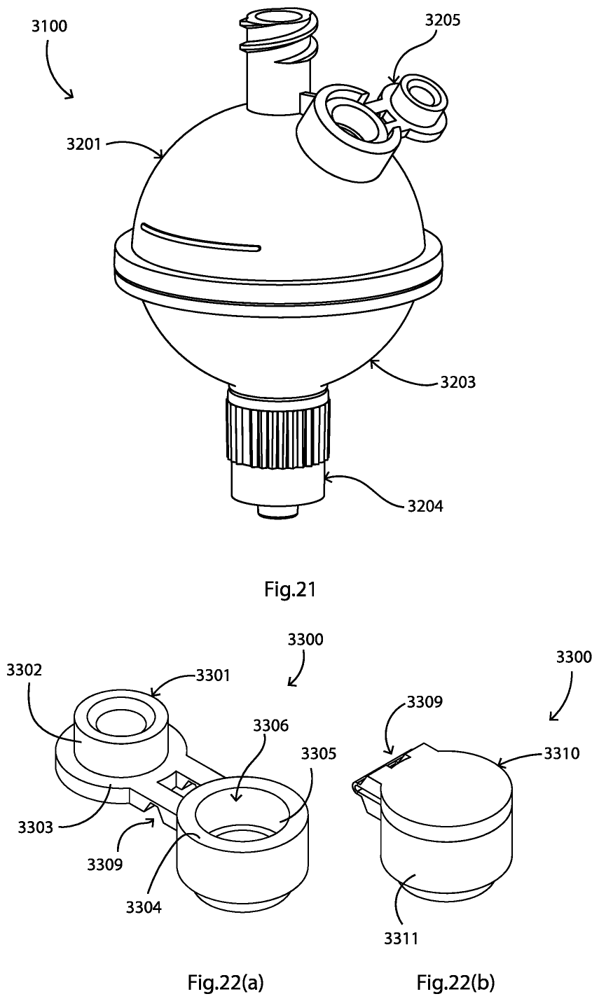

FIG. 19 is a vertical side sectional view of an alternative gas trap device, FIG. 20 is an exploded perspective view of the device, and FIG. 21 is a perspective view of the device FIGS. 22(a) and (b) are perspective views of a priming port of an alternative example in open and closed positions;

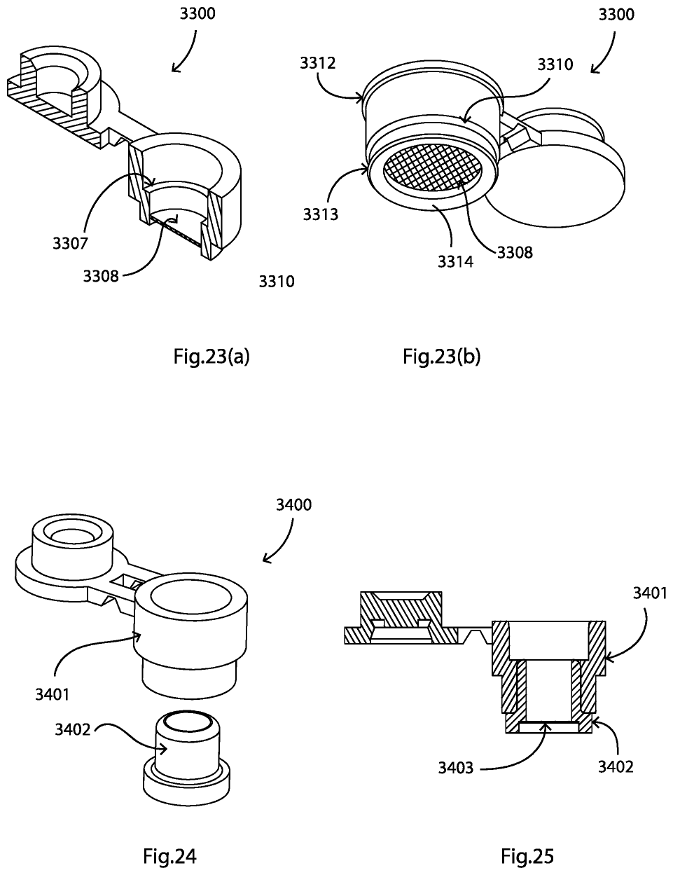

FIGS. 23(a) and (b) are a cut-away perspective view and an underneath perspective view of an alternative priming port;

FIGS. 24 and 25 are perspective and sectional front views respectively of a further priming port;

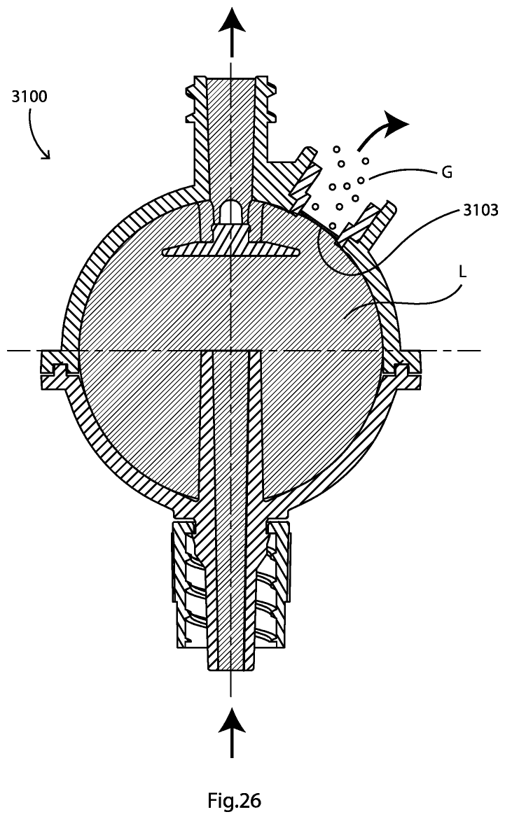

FIG. 26 is a vertical sectional view of the device of FIGS. 19 to 21 in use;

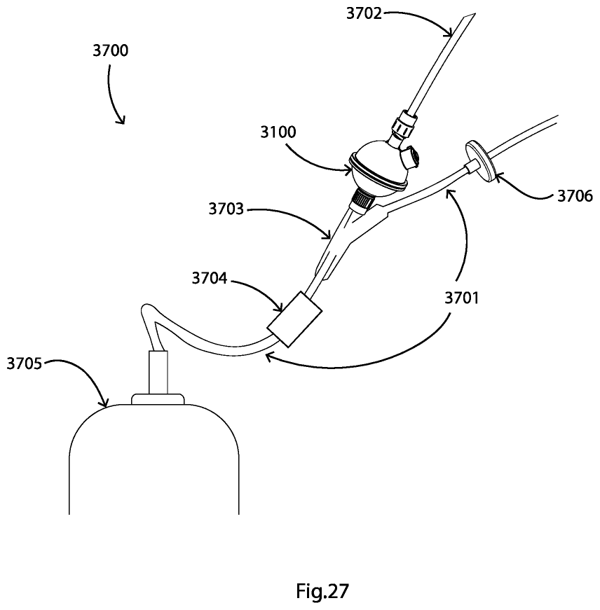

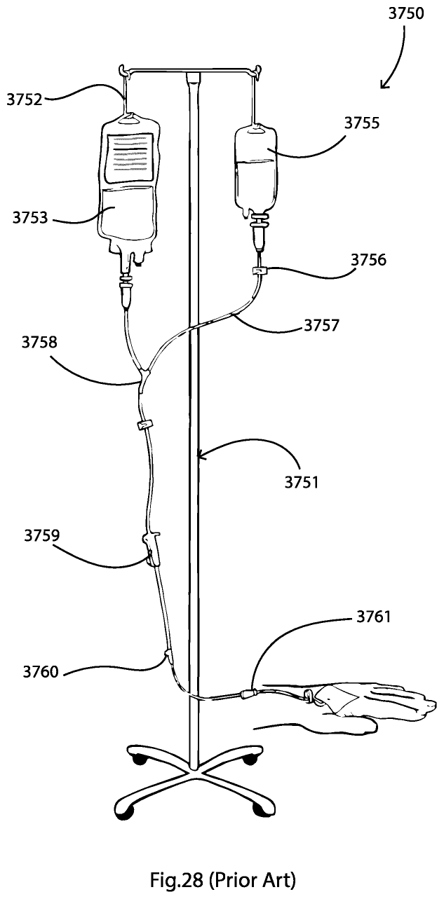

FIG. 27 is a perspective view of a gas trap device of the invention in use in an intravenous line, and FIG. 28 is a diagram showing IV line context;

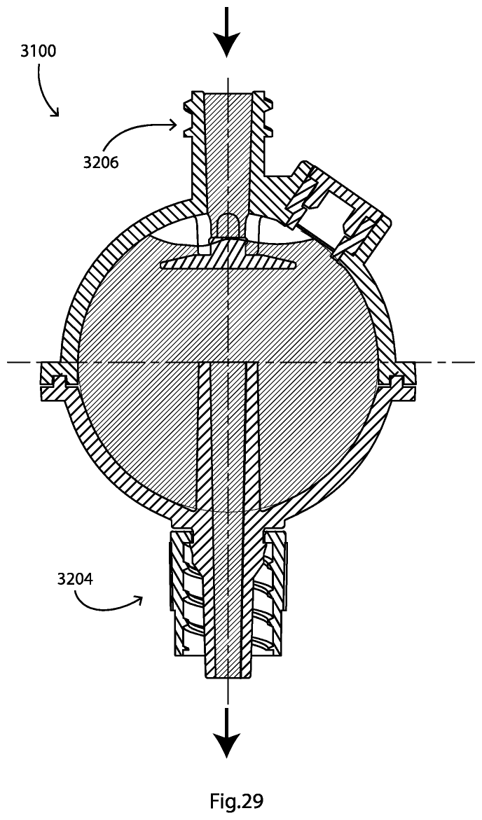

FIGS. 29 to 31 are diagrammatic sectional views showing the gas trap device of FIGS. 19 to 21 in use, with fluid flows illustrated;

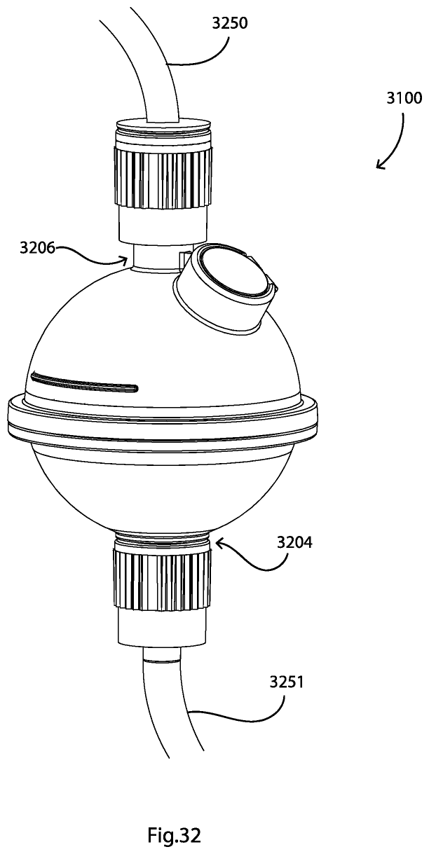

FIG. 32 is a perspective view of the gas trap device of FIGS. 19 to 21 when connected to inlet and outlet lines; and

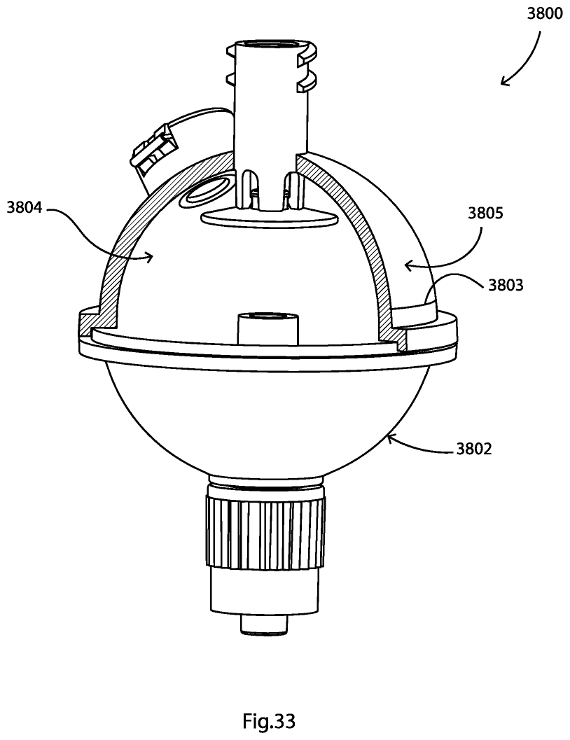

FIG. 33 is a cut-away perspective views of a still further device, having a transparent window,

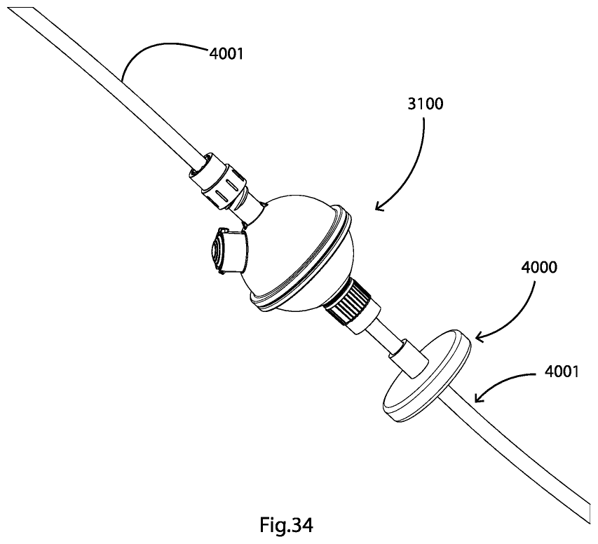

FIG. 34 is a perspective of the device of FIGS. 19 to 21 when connected in series with a particle filter downstream of the device,

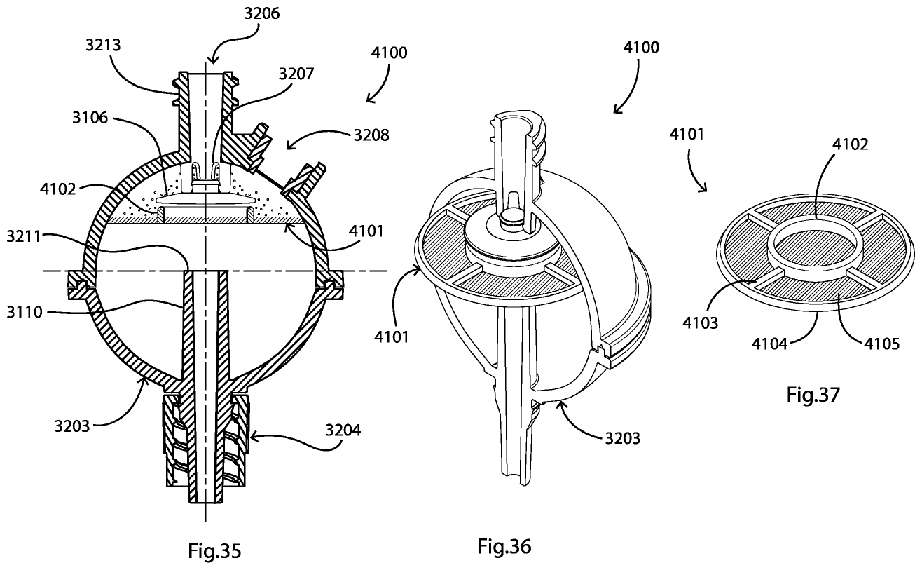

FIGS. 35 and 36 are cut away sectional views of an alternative gas trap device of the invention, in this case including a particle filter within its chamber, and FIG. 37 is a perspective view of the particle filter;

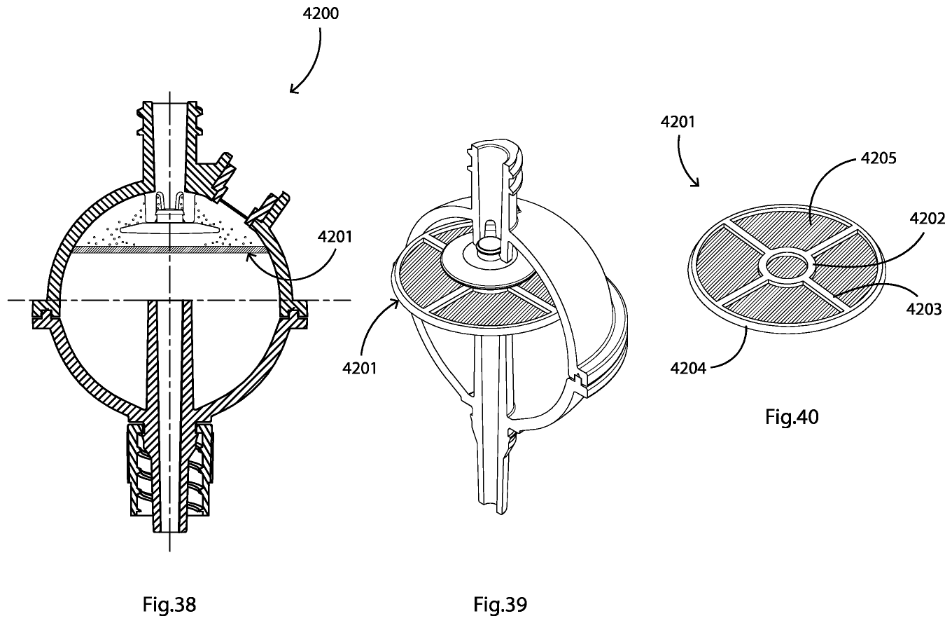

FIGS. 38 and 39 are cut away sectional views of a further alternative gas trap device of the invention with a particle filter within its chamber, and FIG. 40 is a perspective view of the particle filter;

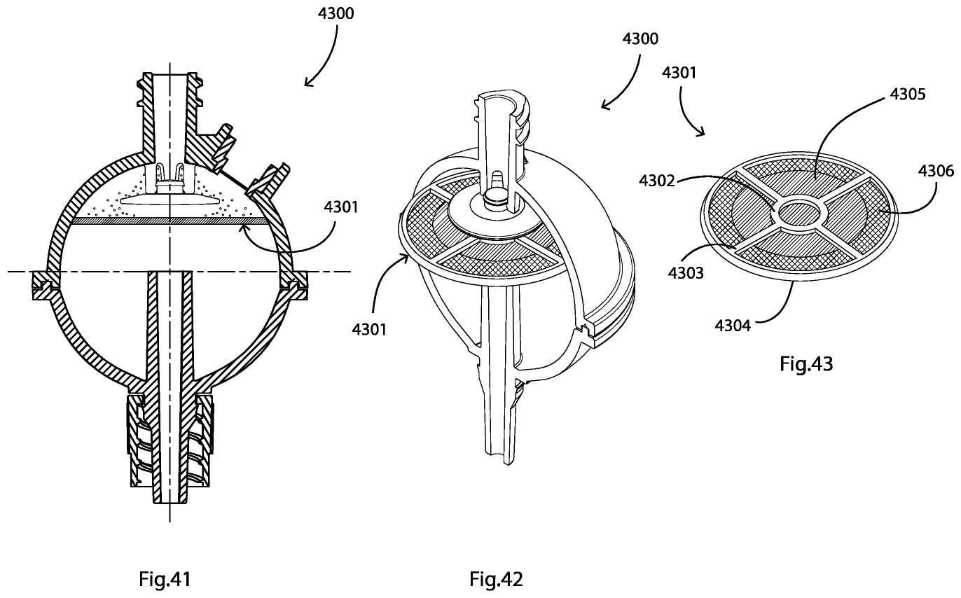

FIGS. 41 and 42 are cut away sectional views of a further alternative gas trap device of the invention with a particle filter within its chamber, and FIG. 43 is a perspective view of the particle filter in which case having an annular region for attraction of particles away from the filter central axis (which in this example is coaxial with the device inlet and outlet);

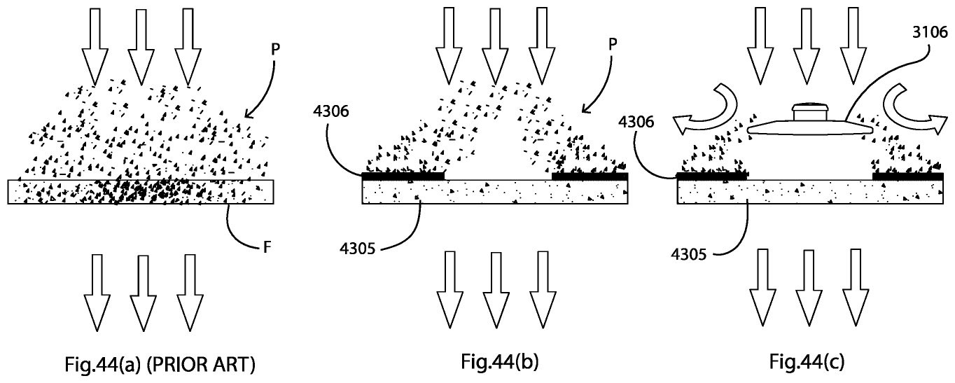

FIGS. 44(b), and (c) are diagrams illustrating operation of the filter of FIG. 43, with FIG. 44(a) showing prior art context;

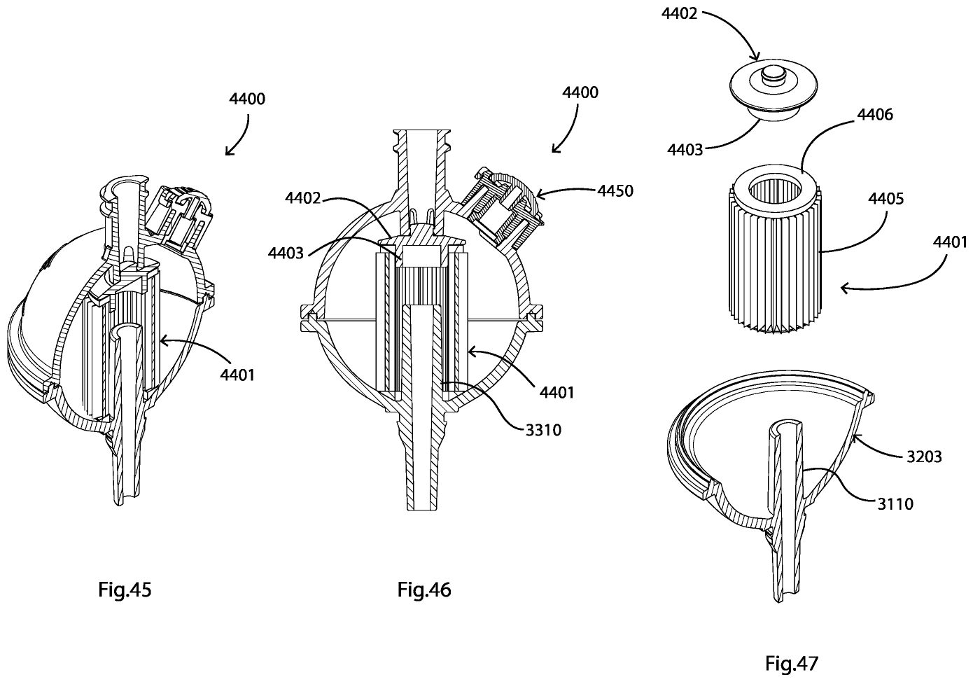

FIGS. 45 and 46 are cut away sectional views of a further alternative gas trap device of the invention with a particle filter within its chamber, and FIG. 47 is a perspective view of the particle filter, in this case having a tubular configuration and extending coaxially and proximally of the outlet conduit;

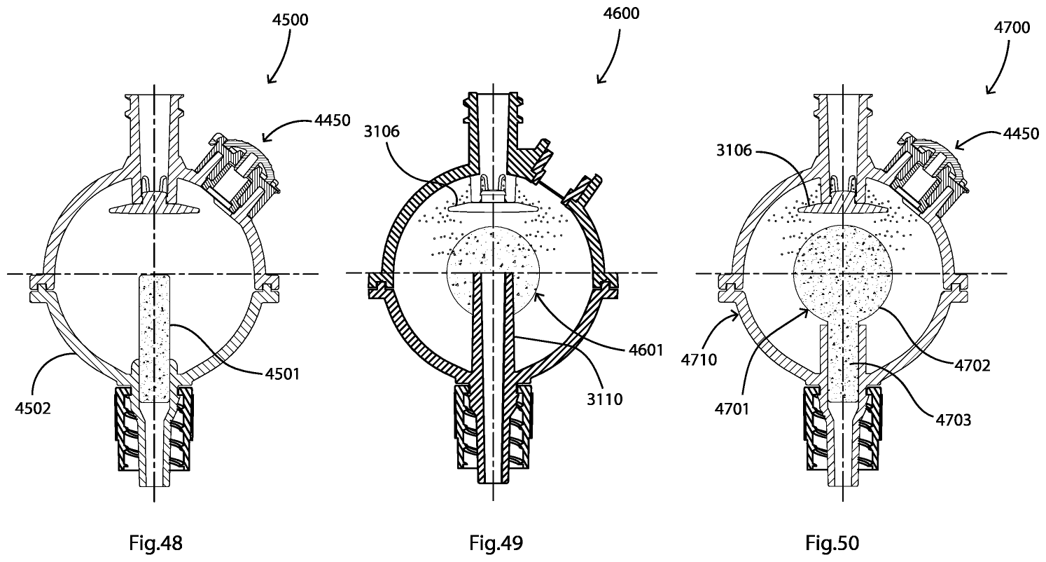

FIGS. 48, 49, and 50 are front sectional views of gas trap devices with different particle filters within their chambers;

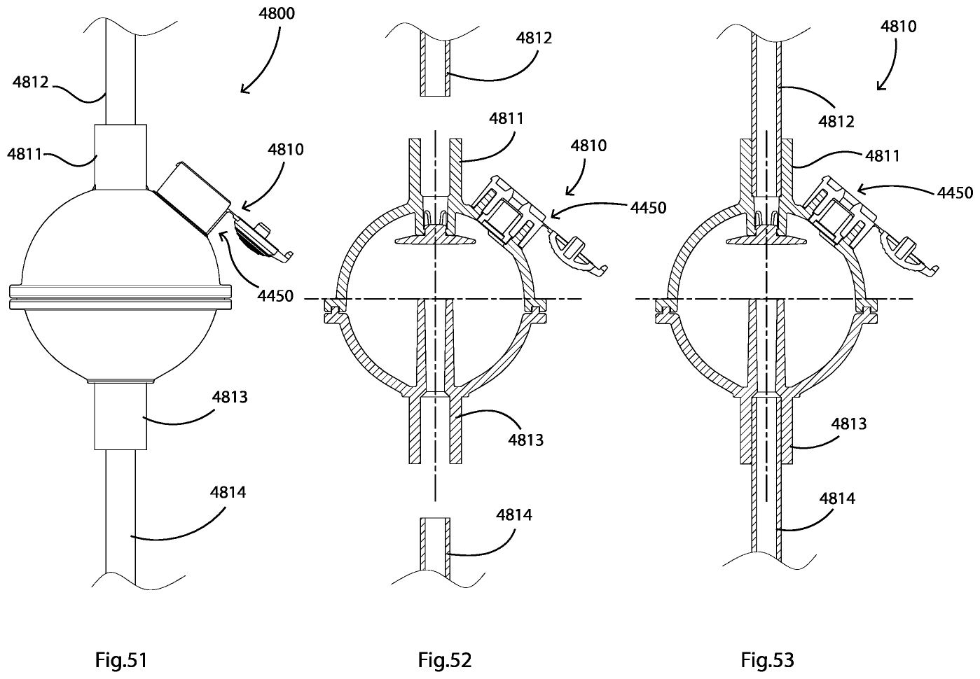

FIG. 51 is a front view of a gas trap device with permanently connected inflow and outflow lines as an integral assembly, and FIGS. 52 and 53 are front sectional views showing connection of the lines to the device inlet and outlet;

54 and 55 are front sectional views showing connection of flow lines to a device inlet and outlet, in this case with the device ports being bonded within the ends of the flow lines;

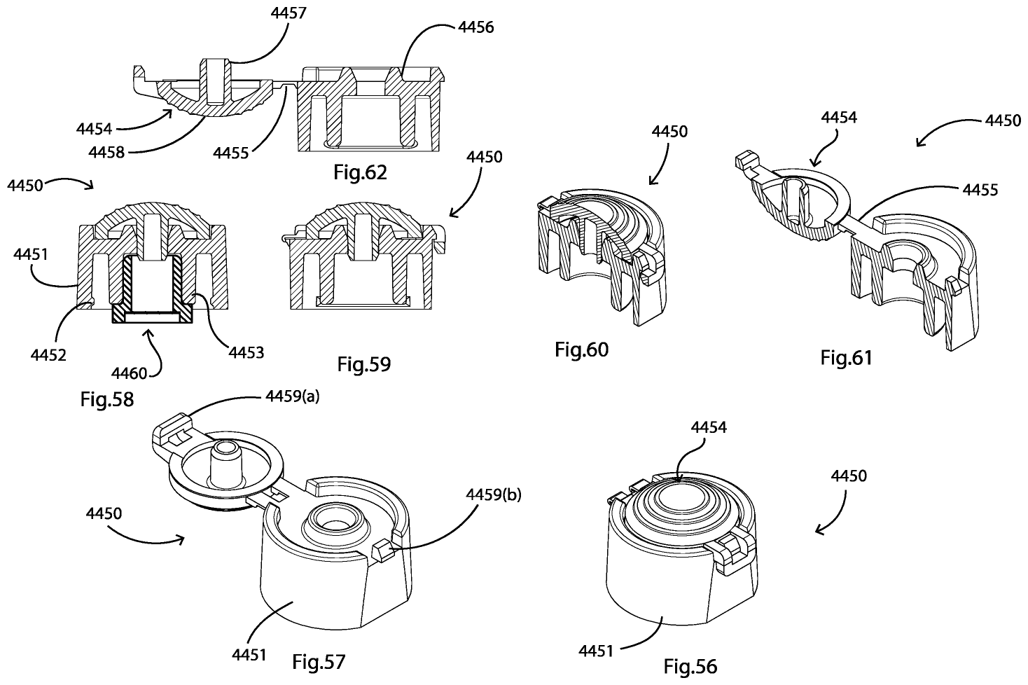

FIGS. 56 to 62 show aspects of a priming port, in which:

FIG. 56 shows the priming port in perspective view when closed,

FIG. 57 shows the priming port in perspective view when open,

FIG. 58 is a front sectional view of the port mounted to a priming port opening rim of a device chamber,

FIG. 59 is a front sectional view of the priming port when not connected to a chamber,

FIG. 60 is a perspective cut-away view of the priming port when closed, and FIG. 61 is a similar view when open, and

FIG. 62 is a front elevational sectional view of the priming port when open; and

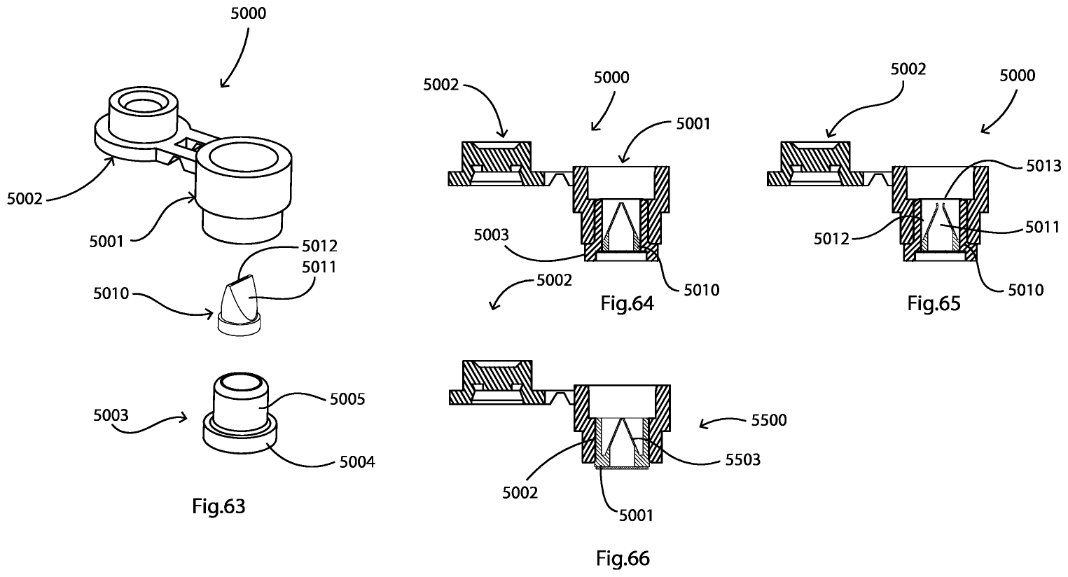

FIGS. 63 to 66 are various drawings illustrating aspects of a priming port with a backcheck valve, in which:

FIG. 63 is a set of perspective views of components of the priming port, showing a port housing and cap, a backcheck valve, and a seat of the valve, and

FIGS. 64 and 65 are front sectional views showing the priming port with the valve closed and open respectively; and

FIG. 66 is a front sectional view of a priming port with a different valve, in this case incorporating a tubular housing.

### DETAILED DESCRIPTION OF THE EMBODIMENTS

Referring to FIGS. 1 to 7 a gas trap device 1 for a medical infusion apparatus is shown. The device 1 comprises a generally spherical chamber 2 formed by a first (top) hemispherical part 3 and a second (bottom) hemispherical part 4. There is an inlet port 10, and an outlet port 15. A priming port 20 extends substantially parallel to a longitudinal axis of the chamber 2 and adjacent to the inlet port 10. The priming port 20 has a tubular body 21 and a sealing cap 22. The inlet port 10 has male Luer features 11, and the outlet port 15 has a Luer lock ring 16 and a female Luer fitting 17. The inlet and outlet ports are in this case coaxial, on a longitudinal axis of the chamber 2.

Referring particularly to FIGS. 6 and 7, the inlet port 10 extends distally into the volume of the chamber 2 and terminates in a diverter 30 which is linked to the inlet port 10 by axially extending circumferential wall segments 33 defining in this example three radial openings 32 for inlet fluid to enter the chamber. One of the wall segments 33 faces in the radial direction of the priming port 20, thereby preventing splashing of liquid towards the priming port 20.

As shown most clearly in FIG. 6 the innermost, distal, surface 31 of the diverter 30 facing away from the inlet direction, is convex. Advantageously, this convex surface 31 is facing towards the outlet conduit 18. The outlet conduit 18 extends into the chamber and terminates with an opening 18(a) at a location approximately centrally within the chamber, and in-line with the chamber's longitudinal axis. In this case the opening 18(a) is exactly central within the chamber, but in other embodiments it may be in a central region such as within 40% to 60% across any dimension of the chamber.

The priming port 20 is non-openable after it has been used for priming, as the cap 22 is a tight friction fit within the body 21, and there is no convenient hand or tool grip surface on the cap. The body 21 includes a priming filter 23, in this case having a 3 μm mesh size across the body immediately below the level of the cap 22 when it is sealed. The filter 23 mesh size helps to restrict flow of liquid out of the priming port while allowing flow of gas out of this port. In some examples the priming filter is of, or has a coating of, a hydrophobic material to contribute to resistance of liquid flow out of the port during priming.

The chamber has, in this example, a volume of about 9.3 ml, and so there is a volume of slightly greater than 3 ml above the level of the outlet port conduit 18. The internal diameter is 26.6 mm and the external diameter is 29 mm. In general, it is preferred that the chamber has a volume in the range of 1 ml to 30 ml, and more preferably in the range of 3 ml to 15 ml.

For IV use, the following parameter ranges are preferred. The inlet port diameter is preferably in the range of 0.5 mm to 6 mm, in one example 3 mm ID. The priming port diameter is preferably in the range of 0.5 mm to 6 mm, and the outlet port diameter is preferably in the range of 0.5 mm to 5 mm range, for example 3 mm ID.

Devices of the invention may be used for other applications such as delivery of fluids on very small scales such as nanoscale volumes for targeted drug delivery. In such cases the above dimension ranges may have lower values as low as 0.01 mm. Other applications may be in manufacturing or fuel systems.

The diverter 30 is in the form of a dish, and has surfaces as follows:
A projection 35 facing proximally into the inlet port 10, in this case co-axial with the inlet port.
An annular concave surface 36 around the projection 35 and facing proximally.
A rim forming a circumferential edge of the diverter 30 with a proximal surface 37 which is sloped radially outwardly and distally from the inlet side (the inlet side being proximal and the outlet side being distal).
On the side facing distally, the convex surface 31.

Referring primarily to FIGS. 6 and 7, an inlet fluid with liquid and possibly some gas flows in an intravenous (“IV”) tube to the inlet port 10 is shown. Flow is redirected through the three openings 32 before entering the main chamber volume. Due to buoyancy and rapid degassing in this uppermost and turbulent zone any gas such as air is separated here. The flow in this turbulent zone is around the projection 35 and is guided by the diverter proximal surface 36 radially through the openings 32. The projection 35 and the annular surface 36 have the effect of turning the flow through an angle greater than 90° with respect to the inlet port axis, and at the same time spreading radially to the extent allowed by the openings 32. The openings 32 have dimensions of 1 mm×3 mm, giving an area of 3 mm². In general, it is preferred that the openings have a size in the range of 0.25 mm² to 10 mm², and in general it is preferred that they occupy an area of less than half of the circumferential area of the inlet port where there are located.

The inlet openings 32 and the surfaces of the proximal side of the diverter form a diffuser to cause turbulence to bring about immediate separation of gas as the fluid enters the chamber. This is especially caused by the rapid change in direction and spreading out through the openings 32, which as shown in FIG. 7, only take up about 25% of the circumference of the inlet port, and in general it is preferred that it is less than 50%.

Large bubbles may be broken up at the edges of the openings 32 arranged about the diffuser, or the large bubbles may deform to squeeze through the openings 32 and remain as one large bubble. At this stage the forced turbulence of the flow into the uppermost region of the chamber 2 increases degassing, a natural phenomenon of mixing fluids, and propagates air bubbles. Due to buoyancy these gas bubbles will remain at the top of the liquid body. The gas entrained in the fluids is separated and the gas phase is created or enhanced at the uppermost region of the chamber.

Flow from the openings 32 may be regarded as being in a transition zone, acting as a filter to remove the gas at the top. The liquid moves around the diverter plate 30 where it settles into laminar flow. This is assisted by the fact that the diverter rim proximal surface 37 is tapered distally and radially.

The predominantly gas-free liquid flows in a laminar manner distally from the diverter plate 30 to the outlet port conduit 18, and more specifically, its opening 18(a). The trapped gas remains in the uppermost section of the sealed chamber.

The device 1 provides an advantageous air phase separation function at the point of entry to the chamber. The composite fluids flowing into the device are treated by three naturally occurring flow zones that control phase-separation between the liquid and gas phases. Immediately upon entry to the chamber, the holes 32 arranged around the inlet acts as a diffuser, redirecting the flow path and forcing fluids to flow under pressure into the chamber where buoyancy forces are accelerated due to the turbulent flow of the fluids. At this point, larger bubbles may be broken up by the edges of the entry holes 32, or the large bubbles may deform to squeeze through the holes and remain as one large bubble. As fluids pass through the smaller openings the velocity of flow is increased and the turbulence is thus accentuated. At this stage the forced turbulence of the inflow into the uppermost region of the chamber increases degassing, a natural phenomenon of fluids that propagates air bubbles. Due to buoyancy these air bubbles rise up from the fluid body and will remain at the top of the fluid body. The air phase is separated and trapped at the air-fluid interface at the uppermost region of the chamber. Then, the diverter 30 creates a slower flowing transition region where the speed of the liquid and shear forces of the water molecules is rapidly decelerated, ensuring gases are removed primarily at the uppermost region of the chamber. As the fluid molecules transition through a shear flow profile into a direct flow profile the liquid becomes laminar and stable before moving into the wider body of fluid. Lastly, the stable, laminar flowing fluid that fills the chamber is allowed to exit from the centre 18(a) of the chamber and is thus predominantly free from bubbles that could result in patient harm.

It is advantageous that the chamber 2 is rigid, and in this case, it has a rim 5 where the hemispherical parts 3 and 4 are joined. This helps to ensure that the full volume is available and that operation is not adversely affected. It is also very advantageous that the priming/venting port is for single use, for permanent closure. The description below referring to FIGS. 9 to 18 gives further examples of arrangements to achieve this. The configuration of the flow diverter 30 is particularly effective at achieving strong initial turbulence at the top of the chamber, helping to avoid gas migrating distally down towards the outlet conduit. The projection 35 into the inlet, and the curved and annular surfaces to cause a bend through at least 90° with respect to the longitudinal axis and the small area of the openings 32 proximally of the diverter help to provide this extent of turbulence at a high level within the chamber. Another advantageous feature is that the diverter is closer to the proximal end of the chamber than to the centre, in this case within the range up to 40% from the proximal end of the chamber to the distal end. This forces the gas-liquid separation to occur in a small, proximal-most, volume providing a large volume for laminar liquid flow to the outlet conduit 18. It is also advantageous in some applications that there is a convex surface facing distally, thereby assisting radial and upward flow of any bubbles which do flow distally of the diverter 30.

### Example Methods of Use

### Primary Flow Method of Use

The following are steps of use of the device 1 for only a single, primary, flow of fluid.
Remove a cap from the top (proximal) connector at the proximal end of the inlet port 10.
Connect a coupler such as an IV line male Luer connector to the inlet port 10.
Ensure that a bottom (distal) cap is in place on the outlet port 15.
Prime the chamber with saline only with the device 1 in its upright position.
Keep the bottom cap tightly sealed and the venting or priming port cap in the open position.
Open a roller clamp on the IV line proximal to the device, allowing saline to fill the chamber fully. The chamber 2 fills because the outlet port is closed.
Once the chamber is completely filled with the saline, close the cap 22 on the vent port 20.
Press firmly to ensure a good seal.
Close a valve such as a roller clamp to stop inlet fluid flow. Open and discard the cap on the bottom, outlet port, of the device.
Connect a male proximal end of the extension IV line to the bottom, outlet, port, ensuring a tight seal.
Open a distal roller clamp and allow IV fluids to run through as normal.

Bubbles that appear in the primary IV line will be captured by the chamber. Up to 3 ml of gas can be captured. If more than 3 ml of gas is present in the device, one should consider replacing the IV line.

### Secondary Flow Method of Use (Referring to FIG. 8)

Remove a cap from the bottom (distal, outlet 15) connector of the device.
Connect a coupler such as a male Luer connector to the female port of a Y-connector 50, ensuring a tight seal. The Y-connector 50 is connected in a secondary IV line 51.
Ensure the IV line is closed distal to the device by closing a pinch clamp 52 on the distal end tubing.
Prime the chamber with a liquid such as saline only with the device in an upright position.
Keep the top, inlet port 10, cap tightly sealed and the venting/priming port 20 in the open position. This causes flow in the secondary line 51 to be diverted upwardly into the chamber via the outlet port 15, and liquid fills the chamber while air is vented through the open priming port.
Open a valve such as a roller clamp on the IV line proximal to device.
Allow a liquid such as saline to fill the chamber fully.
Once the chamber is completely filled, close the cap 22 on the vent port. Press firmly to ensure a good seal.
Open and discard the top cap on the female Luer connector on top (proximal, inlet) port 10.
Connect the male (distal) end of the secondary IV line to the top port 10 of the device, ensuring a tight seal.
Open the pinch clamp 52 below the device and allow IV fluids to run through as normal.

Bubbles that appear in the secondary IV line will be captured by the chamber. Up to 3 mls of air is captured. If more than 3 ml of air is present in the device, consider replacing the IV line.

Bubbles observed in the primary IV line can be readily aspirated without opening the IV system:
Close the pinch clamp below (distal) the Y-connector.
Lower the height of the primary IV bag to make the secondary bag dominant.
Allow the fluids containing the bubbles to flow upwards into the device chamber 2. The bubbles will be trapped in the chamber 2.
Re-position the primary bag as the dominant bag.
Open the distal pinch clamp to resume IV flow as normal.

In other examples the junction distally of device is provided other than by a Y-connector, such as a T-connector.

### Alternative Vent/Priming Ports

### Cap for Hazardous Drug/Toxic Biologics Chamber

We describe below various vent or “priming” ports which have a vent opening and a cap which is simple to put in place to seal the port in various examples of the invention, but either impossible or difficult to remove. This allows initial venting to purge a line but sealing thereafter as a fluid such as a toxic fluid is administered. Where the cap is configured to be difficult to remove it may have a recess for rotation by a tool for removal but cannot be removed by hand. The cap prevents migration of toxic gases to the surrounding environment.

Referring to FIG. 9 a vent port 2100 comprises a cap 2101 which fits into a vent opening 2110, the cap 2101 being intended to seal the chamber opening 2110 in a manner which is difficult to open. The port 2100 can form part or whole of a trap chamber that contains fluids that are intended to be separated from internal and external environments. By placing the cap 2101 into position, the opening 2110 is fully sealed with an air-tight seal and no fluids can pass through. The cap 2101 can be pushed into place, twisted, squeezed or in any other manner forcibly placed into the orifice opening.

In this embodiment the cap 2101 comprises downwardly-depending legs 2106 separated by a V-shaped gap 2103 to provide a split feature that is intended to flexibly deform when pushed into the mating vent opening 2110 of the chamber. The mouth of the opening 2110 has a radially inwardly-directed rim or protrusion 2105 that locks into position with a circumferential groove 2104 in the cap 2101. When fully in place, the downwardly and inwardly angled walls of the legs 2106 of the cap base are in contact with the internal surfaces of the walls of the chamber opening 2110 to form an airtight seal. The top of the cap 2101 has a convex surface 2108 that is intentionally difficult to grasp and thereby reducing potential for removal of the cap by human hand, such that the cap 2101 and the chamber 2111 are forcibly sealed and held together to form a sealed unit.

FIG. 10 is a set of views of an alternative vent port 2200, having a cap 2201 and a vent opening 2210. In this case the cap 2201 has legs 2206 which have outer surfaces tapered inwardly to narrow on the end which is inserted into the opening 2100, and separated by a V-shaped gap 2203. Again, there is a circumferential groove 2204. In this case, however, the top 2208 has a lower profile, with a shallow depression 2209 diametrically across its centre. Also, there are additional circumferential grooves 2215 and 2216 below the groove 2204. The opening 2210 has corresponding ridges 2210, 2211, and 2212 to engage tightly with the grooves 2204, 2215, and 2216.

In other examples there may be a pair of cross-shaped cut-outs akin to the cut-out 2203, allowing more uniform flexibility around the circumference.

As shown in FIGS. 11 and 12 a cap 2301 may have a diametrically extending slot 2302 for gripping by a tool, or a cap 2401 may have a cross-shaped groove 2402 for engaging a tool.

The cap, preferably with a split base, may have any of a variety of suitable shapes and materials. Some materials may be selected for their low or high friction coefficient properties, or elasticity properties, such that the material can distort or flex as required to fit into the opening. The split may be of any of various notched shapes to enable flexible distortion. The material may be required to return to its given geometry once a force is removed.

The cap top may be of a material that is the same or different to the cap base material. The cap top shape may be round, flat, convex, concave or any other geometric shape that is intentionally difficult to grip or pull.

As noted above the cap may have one or more notches that interface with protrusions, such that, when the notch moves past the top edge of the protrusion, the cap is rigidly held and locked into position using friction or surface to surface contact.

For convenience it is practical to have the cap located on or near the device that requires closure. In one embodiment shown in FIGS. 13 and 14 a cap 2501 is attached to the opening 2510 of a chamber 2511 by means of a flexible connector or ribbon 2530, such that the cap can be maneuvered into the opening. The ribbon can be attached to the chamber by bonding, connecting, clipping or moulded in place. The cap 2501 is in this case attached by the ribbon 2530 to the opening by means of a ring 2531 that slips over the opening 2510. The purpose of the ring is to enable the cap to be twisted into place or pushed into place, with the ring rotating to a convenient position and without shearing or twisting the ribbon 2530.

In one embodiment shown in FIG. 15 the ribbon 2530 comprises the ring 2531 at the inner end for engaging the opening and a disc 2533 at the outer end for engaging the cap. These ends are connected by a band 2532 with a narrowing geometry or necks 2534 and 2535 at the inner and outer ends respectively. These form a weak link so that it can easily pulled, either by hand or an appropriate tool, and disconnected once the cap is in its permanent location. The reason for removing the ribbon is to make it less likely that a person can pull on the cap and remove it from its intended permanent location as a seal.

The cap can be placed into its permanent home location to seal the chamber during the priming process and the ribbon section can be snapped off to indicate that the chamber is sealed. In one embodiment the material is flexible, in another embodiment the material is rigid, in another embodiment the material is a bright colour to identify its location.

In a further embodiment the cap is formed with an internal thread that is of the common Luer type, such that it can be rotated and sealed in place on the outside section of a Luer fitting, which is common practice in medical procedure.

In a further embodiment the cap is formed with a push-fit that uses surface-to-surface friction contact to be sealed in place on the outside section of a fitting.

In another embodiment shown in FIG. 16 in a port 2600 a sealing cap 2601 connects to the port opening 2610 by a Luer thread 2605 that mates with a thread 2613 on the outer face of the port, namely on the external surface of the opening 2610. The cap 2601 has a split 2603 on an internal section, the purpose of which is to distort and narrow when the radially outwardly facing cap face 2606 is in contact with the port opening edge 2611. When fully rotated into place the internal cap face 2604 is in contact with the port face 2612, and as the flexible port expands the cap 2601 is trapped on the port opening 2610. By rotating the cap 2601 on the Luer threads it still remains fixed in its permanent location due to the internal split section being trapped by the contacting faces 2604 and 2612, essentially not removable once in place. The cap 2601 has hand-grip knurls 2608 on the outer face to support tightening of the cap device by hand initially.

In another embodiment the cap is attachable by bonding, connecting, moulding or other suitable process such that it is always connected to the device with the open port.

In another embodiment shown in FIG. 17 a cap 2700 has many features equivalent to those of FIG. 20, but in this case the cap 2701 is linked to the opening 2710 by a ribbon 2730. FIG. 18 shows a variation, in which a cap 2800 has a cap 2801 which is similar to the cap 2701 except that it has a pair of narrow knurled grip surfaces 2801 and 2802 separated by a ring 2831 which is integral with the ribbon 2830.

In another embodiment the cap is seated in a normally closed position within a housing on the chamber of the device, the cap formed with a geometry such as cuts, grooves or narrowing features that can deflect when a force is applied to the top of the cap in a downward direction. The deflecting cap under force by hand can be depressed into the body of the housing chamber and the distal features of the cap engage against a retaining surface that prevents the cap from pushing through an opening beneath, instead the cap returns to its set position due to stored kinetic energy and material elasticity.

### Advantageous Uses

Chemotherapy is a toxic drug that is commonly used to provide medical treatment for a broad range of cancers. It is most often delivered in intravenous format as a liquid drug that is infused directly into the vein of a patient, either in a hospital, clinic or home environment. Hazardous drugs can include a broad range of agents other than chemotherapy, including toxic biologics or drugs with carcinogenic, mutagenic and antineoplastic properties, all of which are harmful to human health.

It is preferable to administer toxic biologics or hazardous drugs (HD) in a closed system that does not allow exposure of environmental contaminants into the line nor exposure of the toxic substances to the surrounding environment. During intravenous (IV) infusion of the drug, trace amounts of the toxic fluid can escape the IV system and settle on surrounding surfaces. These can be in the form of liquid and/or gas and HD are observed to vaporize at room temperature and when exposed to ambient atmosphere. In modern healthcare, devices know as Closed System Transfer Devices (CSTD) are being utilised by healthcare workers to ensure HD are administered without exposure, to ensure the safety of both the patient and the healthcare worker. Adverse outcomes from exposure to HD in liquid or gas format can result in a range of health problems, including reproductive issues such as reduced fertility and increased risk of miscarriage, as well as dizziness, nausea, cancer and death. Repeated micro-dose exposure over long periods of time is shown to result directly in harm to the health of the healthcare workers in proximity to toxic biologics or hazardous drug infusions.

During administration it is not uncommon for the IV line to be opened to aspirate bubbles from the HD infusion lines as those bubbles pose a subsequent risk to the health of the patient. In removing air bubbles, or toxic vapour bubbles, the healthcare worker is placing their own health at risk. It is evident that devices to reduce exposure can have a positive impact on healthcare delivery.

Maintaining the closed chamber is important to protect healthcare workers and their patients from unknown or unintended contact with the chemotherapy or toxic agent that is flowing through the IV. In the setup of the IV, such as priming, where entrained air in the IV tube is expelled to ensure correct and safe performance of the procedure, it is necessary to have an open port to expel the air. Where a device is being used to purge problem bubbles from IV lines, in which the chamber is intended to retain the liquids and gases, the venting port is used to expel the air. The purpose of the open venting port is to allow air to escape the chamber so that a required volume of fluids can enter the chamber. Once the chamber is filled with the required fluids it is then necessary to seal the chamber so that the drug is not exposed in an unsafe environment such as at the bedside. Where blood or hazardous drugs are being administered, it is often preferred and safer to ensure that the chamber cannot be easily opened, to ensure no risk of exposing the toxic drug.

As such, a method to ensure a reliable seal is in place would potentially reduce opening of the priming port and hence the chamber. Preferably, the seal cannot be opened or removed by hand. Such a device, when fixed into position, would provide an airtight seal to ensure that no liquids or vapours would be able to transmit in or out of the chamber.

The seal can be a cap, can be push fit, rotation or latch mechanism, or any other mechanism, such that it can be placed into an active position and not removed easily. The cap may be fixed in place, or it may be selectively sealable such that it can be opened to enable critical functions to be performed. It may seal off passage of fluids, vapours of environmental contaminants by press fit contact between mating surfaces, or it may utilise a sealing ring or membrane device.

### Alternative Embodiments

Referring to FIGS. 19 to 21 a gas trap device 3100 is hereby disclosed that is intended to be applied to an IV line, or other fluid line, to remove air-in-line while enabling IV drugs, or other fluids, to flow freely. It functions by allowing a fluid to enter its chamber, then redirecting flow such that air and liquid are pushed into a turbulence region where degassing is accelerated, the phases of air and liquid then separating and the air rising to the uppermost region under buoyancy forces. The device comprises a diverter plate that is fixed about the inflow tube entry point, to direct flow towards a transition and laminar flow region at the outermost region of the chamber of the device, away from the centre of the body of fluids. The operation of the device 31 is therefore essentially similar to that of the device 1.

The device chamber houses an exit port that draws liquid from the centremost region of the body of fluids, this liquid having been substantially stripped of problem air bubbles.

The structure of the device 3100 has many features in common with those of the devices of the embodiments described above. In this case, the device 3100 has generally spherical chamber with a top hemisphere 3201 and a bottom hemisphere 3203, and both defining an internal chamber volume 3104. There is a priming port 3208 which extends radially rather than being parallel to the inlet port (3206). The device 3100 comprises an inlet port 3206, inlet (diffuser) holes 3207, a diverter 3106, the venting or priming port 3208 with an insert 3205 having a cap. An outlet port 3204 is linked with an outlet conduit 3110 with an intake end opening 3211 at the spherical centre of the chamber. The outlet port 3204 has an outlet end 3210. The outlet conduit 3110 defines a channel 3112 together with the port 3204. There are Luer connections, male 3212 and female 3213 for the device. The diverter 3106 has a proximal surface 3106(a) which is tapered to extend radially and distally. It therefore contributes to the transition to laminar flow of liquid radially into the chamber in the inlet (proximal) end of the chamber 2. It therefore plays the same role as the surface 37 of the diverter in the device 1, and the same comments apply. In this case, however, there is no inner proximal surface which is splayed radially and proximally and hence the degree of change of direction of the inflowing fluid is only 90°. Advantageous aspects of the diverter of most embodiments are that:
They are close to the distal end of the inlet, preferably attached to the inlet port. In general it is advantageous and preferred that the diverter proximal surface be at or less than 40% of the longitudinal dimension (between inlet and outlet) from a proximal-most end of the chamber. It is more preferred that this range be 30%, and even more preferred that it be 20%.
The proximal surface of the diverter is located to guide flow out of the inlet diffuser openings (32, 3207), being at or at least adjacent to these openings, preferably less than 5 mm from them in the longitudinal direction, more preferably less than 3 mm.
The diverter proximal surface (37, 3106(a)) which is tapered radially and distally, contributing to effective flow of fluid into the proximal region of the chamber, contributing to the laminar flow described above.

The device 3100 comprises only six parts, the parts being arranged to form a closed system aspirator device that is intended to remove bubbles of gas such as air from flowing fluids in any orientation and capture and retain these bubbles without restricting flow of fluids through the device. It is intended to be used with gravity or pump-driven IV infusions, or other flow procedures, where air bubbles are harmful or disruptive. The top hemisphere 3201 is optimally formed by injection moulding or other method suitable for production, formed by plastics with a set of features further described below. The top hemisphere 3201 is attached by means of snap fitting of a proximally extending rim into a distal end of the inlet port 3207. In other examples the diverter is mounted by press fitting or bonding. The diverter 3106 is optimally formed by injection moulding or other method suitable for production, formed by plastics with a set of features further described below. The vent 3208 is attached to the top hemisphere 3201 by means of snap fit, press fit, bonding, or other manufacturing method. The vent insert 3205 includes a membrane 3103 that aspirates air and retains fluids by means of a hydrophobic coating, and the membrane may be fixed in place by over-moulding, bonding with glues, ultrasonic welding, heat welds or any other method of attaching or otherwise holding the membrane in place within the vent 3208 or chamber of the device 3100. The bottom hemisphere 3203 is optimally formed by injection moulding or other method suitable for production, formed by plastics with a set of features further described below. The bottom hemisphere 3203 is attached by means of snap fit, press fit, bonding, or other manufacturing method to the rotating male Luer ring 3212, which remains fixed in place after assembly or manufacturing. The hemispherical chamber parts 3201 and 3203 are joined permanently together at circumferential flanges 3116 and 3117. The joining process provides a water-tight seal. Preferably, the joining method is ultrasonic welding but may be by means of snap fit, press fit, bonding, gluing, spin welding, heat welding, 3M welding, moulding, or any other manufacturing method suitable to join two parts together to form a chamber. This disclosure covers this combination of parts, or any other embodiment of this type of device that is intended to remove bubbles of air from flowing fluids in any orientation and capture and retain these bubbles of air without restricting flow of fluids for the purpose of aspirating air automatically from intravenous drugs and trapping said air within the chamber as a so-called closed system aspirator or closed system administration device.

Referring to FIGS. 22 and 23 a priming port 3300 is shown, being suitable to fit into a venting rim of the chamber and this may be part of a gas trap device of any example described herein. The priming port 3300 is shown in initial open position (FIG. 22(a), and in a closed position (FIG. 22(b)). It is manufactured from a plastics material such as polypropylene, polyethylene or any other plastic that can be moulded or otherwise formed into the required shape, with a rigidity and flexibility that retains its overall shape but can be malleable and deformed for press fitting into a chamber. The priming port 3300 comprises a hinge 3309 that is flexible and allows a cap 3301 to flip closed into the body 3311, formed as one device or with connecting or mating parts for the cap and body. In a preferred embodiment the port is formed from a single injection mould and the cap 3301 and body 3311 are connected via an integral hinge 3309. When closed, the cap 3301 forms a tight seal with the body 3311 due to interference fits between a cap external surface 3302 and the body internal surface 3305, and a cap surface 3303 and a body surface 3304, or the cap surface 3301 and the body surface 3307, or any combination of the aforementioned surfaces. The cap 3308 has no external grip features and forms a fine seal with the body 3311 such that it is difficult to grip or reopen the cap once it has been closed.

The priming port 3300 forms an open port 3306 in the open position such that liquid, gas, vapour or solids can pass through. In a preferred embodiment fluids or gas can pass through. In a preferred embodiment the port 3300 comprises a membrane that is selectively permeable, and in some examples the membrane has a hydrophobic coating applied to its surface to prevent or reduce liquid from passing through and allowing air only to pass through. The membrane 3308 may be fixed in the lower section of the port 3300. The body has features such as undercuts 3307 and steps 3310 that prevent the cap from commuting or falling when the cap is placed into a preferred position or mated with a surface. The port 3300 comprises snap fits 3313 that lock into place when pressed into a mating location on the device housing such that the cap cannot be pulled back out of its position once it has been pressed into place. In another embodiment the port is bonded to the chamber. In a preferred embodiment the port is press fitted into place. In another embodiment the filter membrane is bonded. In a preferred embodiment the filter membrane is held in place by the port that sits over it in its preferred position. In a preferred embodiment the filter membrane is located in line with the proximal surface 3314 of the port housing. In another embodiment the filter membrane is located in a position that is away from the surface 3314 of the cap housing and forms a chamber or tube.

Referring to FIGS. 24 and 25, in another embodiment a priming port 3400 has many features in common with those described above, but in this case a membrane 3403 is housed in a port body 3402 and is inserted by means of press fit into the body 3401. The membrane and cap function as previously described.

The purpose of the priming port is to ensure that gas such as air is initially discharged from the chamber but the chamber can then be sealed permanently. The vent is activated by hand by the worker and can be readily manipulated into the closed position. For convenience and one-handed operation, the vent is positioned in the upper region of the device when the device is in the upright position and the inlet port is at the highest point. The port is normally open to facilitate the venting of initial air from the chamber. Once the chamber is filled with liquid then the cap is permanently closed to prevent the release of air or vapours once the device is operational. In another embodiment the device can be selectively openable. In another embodiment the port houses a filter of charcoal or other material that is intended to filter fine particles of gas and clean or neutralise the harmful components of these gases. In another embodiment the port is an open port that comprises a gas filtration material. In another embodiment the gas filtration material is fixed directly to the device chamber.

Referring to FIGS. 26, 27, 29, 30, and 31 use of the device 3100 is shown in illustrative examples, together with a diagram in FIG. 28 showing context.

### Back-Priming

A method of clearing the normally entrained air from the device attached to the IV line is described, which is of particular importance when a secondary or piggyback IV setup is used. In one method the device is attached to a connector on the IV line, which can typically be a Y-connector or Luer connector or any other type of connector, the male connector on the distal end of the device connecting to a female connector on the IV system.

A common arrangement of IV uses a primary and secondary combination where two or more lines are connected together to deliver one or more drugs to a patient, using gravity or an IV pump to generate flow. FIG. 27 shows an arrangement 3700 for IV pump setup where a primary line 3701 has a Y-connector 3703 that is used to connect a secondary line 3702. FIG. 28 shows a prior art arrangement 3750 for reference and ease of understanding. In this diagram the infusion set 3750 comprises a stand 3751 with an extension hook 3752 supporting a primary infusion bag 3753 at the outlet of which there is a primary set 3754. There is a secondary or “piggyback” infusion bag 3755 at the outlet of which there is a clamp 3756 linked with secondary tubing 3757. Downstream of both bags there is a clamp 3759 linked to a port 3760, in turn linked with a patient canula 3761.

This arrangement is sometimes called “piggybacking”. The primary line typically delivers a saline solution from a reservoir bag into a patient as per normal IV infusion. Where an IV pump 3705 is used, the primary line is engaged by the pump to control flow rate of the IV fluids. The primary line 3701 may have components attached to it to facilitate flow, such as a pinch clamp 3704 to stop flow or a backcheck (one-way) valve 3706 to stop fluids from the secondary line 3702 from travelling into the primary line 3701 in the wrong direction, towards the reservoir. The secondary line 3702 is typically used to administer drugs which may be hazardous, these drugs being medicinal for the patient. The Y-connector 3703 has a port to which the secondary line 3702 is attached, usually by Luer connection. Due the nature of the drugs in the secondary line 3702, these drugs being volatile in nature, increased levels of degassing are observed. As such, it is useful to include the device 3100 at the distal end of the secondary line 3702 which connects to the Y-connector 3703 of the primary line 3701, the purpose being to capture air from the secondary infusion line 3702. In normal practice the secondary infusion line 3702 is primed by back-priming saline solution from the primary line 3701 such that the saline solution travels back up the secondary line and expels air back into the secondary reservoir (such as the reservoir 3755) in FIG. 28). This method is referred to here as back-priming the line. Once the secondary line is primed, the drugs can be alternately selected by the medical professionals as required.

In practice, sometimes it is necessary for the secondary IV line to be prepared at the bedside or hospital department. A priming method including an gas trap device 3100 is described. The purpose of including a gas trap device on the IV secondary or piggyback setup is to remove air bubbles from the secondary IV line. The priming of the secondary line as previously described can be undertaken with the device 3100 connected to the Y-connector 3703 as shown in FIG. 27 (3700). With the device 3100 in place on the Y-connector 3703 fluids can be directed to flow into the device 3100, which is connected to the Y-connector 3703 and the secondary line 3702 which is further attached to the proximal connector on the device (3100) by means of Luer connection. The venting port cap 3205 can be open or closed while back-priming through the device. The benefit of keeping the venting port cap open during back-priming is that the air in the chamber can be easily expelled as the chamber fills with fluids, which flow up through the distal port end 3210 and into the chamber 3209 through the intake end 3211 of the outflow tube. As such the flow is reversed using primary fluids to fill the chamber. As the chamber 3209 is filled with fluids the air is expelled and the fluids continue to travel up into the diffuser openings 3207, into the inlet port and escape to atmosphere or into the secondary line that is connected via Luer connection 3213 through the outlet port opening 3210. The venting cap 3205 can be closed to seal the chamber.

Once the secondary IV line is primed, flow can be reversed to flow in the ‘normal’ direction, from reservoir to patient, and be used to deliver drugs as intended. The gas trap device of any embodiment described herein or otherwise intended to remove air from flowing fluids when attached to the distal end of the secondary line via a Y-connector on the Primary line, functions as intended to remove and trap air bubbles from the fluids flowing through it.

In another embodiment, the chamber 3209 of the device 3100 is sealed as a first step in the back-priming process by closing the venting cap 3205, the purpose being to ensure a closed system while priming is taking place. In this scenario, air present in the device chamber 3209 does not need to vent through the open port 3208 and can be expelled fully from the chamber through the holes 3207 into the secondary line and secondary reservoir or vented to atmosphere. In another embodiment the device has an open venting port. In another embodiment the device has an open venting port with a filter media to prevent fluid loss and facilitate air venting. In another embodiment the device has a closed venting port. In another embodiment the device has a selectively openable and closable mechanism that can be activated to open or close a venting port.

In another embodiment the device has a selectively openable and single-use, closable mechanism that can be activated to seal a venting port. In another embodiment the device does not feature a venting port but does form chamber. In another embodiment the device is positioned distal to the Y-connector as a component of the IV line, such that it can capture air from fluids of any line attached proximally to the device. In another embodiment the device is positioned proximal to the IV pump as a component of the IV line, such that it can capture air from fluids of any line attached proximally to the device. In another embodiment the device is positioned proximal to the end of the line as a component of the IV line, such that it can capture air from fluids proximally to the device. In another embodiment the device is positioned at any location on any fluid tube, such that fluids can flow into the device from any direction and the device can be vented or can capture air from fluids that flow into the device.

In practice, sometimes it is necessary for the secondary line to be prepared at the bedside or pharmacy department. In this case the device (3100) may be attached to the secondary line distal end and primed normally, as previously described, with fluids, such as saline, biologics or drugs, from the secondary reservoir flowing down the IV line and into the device (3100), which is connected via the Luer connection (3213) on its proximal inlet port (3206) as previously described. The device outlet port (3204) open port can be open, capped or attached to extension tubing via Luer connection (3212), or additional tubing, Y-connectors, cannula or any other suitable IV infusion system for the purposes of delivering the fluids from the reservoir through the device towards a patient.

Referring to FIG. 29 for gravity infusions using secondary or piggyback systems, the primary infusion line is primed as normal with fluids from the reservoir, normally saline solution, used to flush air from the primary line. Once the primary line is attached the secondary line is attached at the Y-connector and the height of the bag is switched so that the primary reservoir is dominant and fluids flow into the secondary line. The purpose of this is to expel all air up through the secondary line into the secondary reservoir. A method of back priming with the device 3100 placed between the secondary line distal end and the primary line Y-connector is described, where the purpose of attaching the device at the priming stage is to expel all air from the device using fluids from the primary line. This method follows the same steps as previously described to back-prime the aspirator as an attached component of the secondary or piggyback setup. This method can be used for both IV pump and gravity based IV infusion systems and is beneficial in practice to improve safety and performance of IV systems that deliver secondary drugs that are gaseous by nature, or where is reasonably considered useful by clinical professionals.

In normal practice, where a single infusion line is being used, the device 3100 can be primed to fill the chamber with fluids as has been previously described. Fluids flow in the direction shown, from the proximal inlet end to the distal outlet end. Air evacuates the chamber through an open port into the surrounding ambient atmosphere.

As shown in FIG. 29, once the device chamber is primed and full of liquid, and residual air is evacuated, the chamber can be sealed by closing the cap of the vent port, which provides an air-tight seal. Fluids, including saline, drugs or biologics, flow through the device in the direction shown, from the reservoir towards the patient. As drugs or other fluids are flowing through the chamber the air phase is separated as previously described and the air settles in the uppermost region of the chamber, regardless of orientation. The priming port filter membrane, which has a hydrophobic coating to repel liquids, ensures that the liquid remains in the chamber, the air being free to transmit to the cavity within the cap housing or to remain in the device chamber. The fluids that are predominantly free of air are able to flow freely through the device and exit the chamber of the device by the outlet port in the direction of the patient.

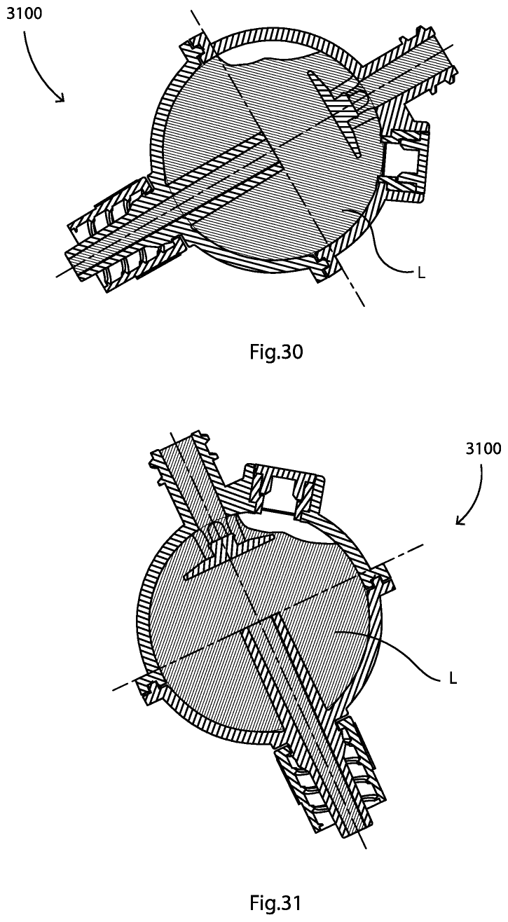

As shown in FIGS. 30 and 31, the device can be rotated through any orientation and the buoyant air phase will remain in the uppermost section of the chamber due the upwards pressure from the fluid body. As air enters the chamber it is diverted to the outer edges immediately and separated through buoyancy in the turbulent regions at the inlet holes. As the device is rotated or moved, for example by ambulatory patients or for convenience of the clinician, any air remains in the chamber and the overall system remains closed to the outside atmosphere due to IV lines connected on both distal and proximal connectors of the device. FIG. 32 shows the device 3100 connected to lines 3250 and 3251.

Referring to FIG. 33, a device 3800 is shown that includes a transparent section of the surface of the material of the device, which may be formed optimally from plastics material, or by any other method or material. In production, a rough surface finish 3802 is mechanically or otherwise obtained that controls or limits the view into the device. In practice, the nurse will prime the device in the upright position with the venting port at the uppermost position. To facilitate priming the material of the device will be a clear plastic that allows the air liquid interface to be readily observed within the chamber. To further encourage the nurse to prime the device in the preferred position with the venting port in the uppermost position, a viewing window 3804 can be created that shows a preferred uppermost section of the internals. The remaining portion of surface area 3805 can be treated to reduce or limit visibility such that the nurse is encouraged to orient the device into the preferred position to observe the internal and to aid visibility of any air during priming or functional performance. A defined grade of colouring, roughness, frosting, transparency can range such that the device may be completely or partially obscured in the preferred locations. It is a preferred embodiment the uppermost section that equates to less than 45% of the total internal liquid volume is 100% transparent and the remaining 55% below this will be semi-transparent, with visibility still possible, but that portion of the device surface obscured due to a mechanically derived surface finish. In another embodiment the surface is dimpled to obscure certain regions. In another embodiment the surface is roughened to obscure certain regions. In another embodiment the indicator line (803) that is formed as a negative or positive indent on the material surface during or after production by moulding, etching, painting or transferring a mark by any other means, the purpose of this mark or indent being to show the safe use levels of the internal fluids to inform the clinician of the volume of air in the device. This mark or indent line 3803 can be used to indicate the transition from fully or substantially transparent plastic material to frosted or otherwise obscured plastic material, including other materials or coverings, to enable the clinician to view or otherwise identify or indicate the internal air fluid interface.

In another embodiment the internal surface of the chamber of the device is coated with a surface treatment that has chemical, medicinal properties to enhance the function of the device when used with certain drugs or biologics, for example, the coating could be a Heparin or other agent that acts against clotting of blood, or the coating could be an agent formulation that reduces propagation of bacteria on the surface of the device or within the fluids that pass through the device. The coating or surface treatment may be applied to one or more parts on the internal or external or both surfaces of the device, and it may be applied to components or features of the device selectively, for example the diverter, extending from the inlet tube to the outlet tube. In one embodiment the surface treatment is applied by gas, vapour, bonding, coating, pasting or otherwise treating the surface with a chemical, water-based or oil-based solution. In another embodiment the surface treatment is applied in the manufacturing phase. In another embodiment the surface treatment is applied to individual components, select surfaces or entire surfaces prior to assembly. In another embodiment the surface treatment is applied to components, select surfaces or entire surfaces after assembly. In a preferred embodiment the surface treatment is a chemical anticoagulant drug, such as Heparin, used to reduce clotting in blood that flows into the chamber and through the device. In a preferred embodiment the surface treatment is an anti-bacterial chemical agent, such as Hypochlorous Acid, Hydrogen Peroxide or other agent, used to reduce or eliminate the number of bacteria that can form on the surfaces of the material or in the fluids that flow into the chamber and through the device.

### Discrete Particle Filter

Referring to FIG. 34 a flow assembly for medical infusion or other applications comprises the gas trap device 3100 (or one of any embodiment) connected in series with a discrete particle filter 4000, providing a flow in a line 4001 towards a destination such as a patient interface. The particle filter is preferably downstream of the device but may be upstream.

By placing an air trapping or aspirating device in series with a particle trapping or filtration device, the dual risks of air embolism and solid thrombus infusion can be simultaneously reduced.

Integrated Particle Filter, within the Gas Trap Chamber

Referring to FIGS. 35 to 36 a gas trap device 4100 has many components in common with the device 3100 and like parts are given the same reference numerals. In this case there is a particle filter 4101 extending across the full cross section of the chamber 3203 distally of the inlet 3206 and being closer to the diverter 3106 than to a centre of the chamber 3203. The filter 4101 comprises a frame supporting a mesh 4105, the frame having a central circular hub 4102 from which extend four equally spaced radial arms 4103 terminating in an outer rim 4104. The rim 4104 is formed with, or bonded to, the chamber wall internal surface. The hub 4102 extends proximally to surround the inlet axis and act as a barrier to flow of gas bubbles towards the centre of the filter 4101. Advantageously, the device 4100 acts to trap particles in addition to trapping gas. This can be very important for applications where unwanted particles arise such as crystals which form in some medicinal liquids, or indeed debris particles from surfaces upstream of the device.

Advantageously, the 4102 limits movement of particles that have been directed to an outside area of the filter, allowing mainly liquid to pass into the axially-central region between 3106 and 4101, where it is transmitted through the membrane 4105. This advantageously improves flow through the device.

A variation of this embodiment is shown in FIGS. 38 to 40, showing a gas trap device 4200 which also has a particle filter in about the same position as the filter 4101. In this case a frame of the filter has a central hub 4202, spokes 4203, a rim 4204, and a mesh 4205. In this case the hub in entirely co-planar with the remainder of the filter frame, allowing more space for flow towards the filter. The membrane 4105 may in some examples be of porous fabric or charcoal.

Referring to FIGS. 41 to 43 a gas trap device 4300 also has many components similar to those of the device 3100 and again like parts are given the same reference numerals. In this case there is a particle filter extending across the chamber across the inlet axis and being normal to it. The filter 4301 comprises a filter mesh 4305 on the proximal side of which there is an annular layer 4306 of charged material to attract particles. The mesh 4305 is supported on a frame having a hub 4302, spokes 4303, and a rim 4304. FIG. 44(a) shows a normal prior art flow of a fluid towards and through a filter, with flow being preferentially directed towards the centre due to the fluid mechanics. This tends to have the effect of clogging the filter and rendering it ineffective after a short time. However, as shown in FIGS. 44(b) and (c) in this case the flow is preferentially away from the centre due to both the charged coating 4306 and the effect of the diverter 3106. This presents a much larger effective filter area and provides for the filter to have a relatively long effective life.

Other configurations of chamber particle filter are possible. For example, FIGS. 45 to 47 show a gas trap device 4400, which also has many parts in common with the device 3100. The device 4400 has a priming port 4450 which is described in more detail below with reference to FIG. 50. In this case a particle filter 4401 comprises a corrugated tubular wall 4405 extending around the conduit 3110 and proximally towards a deflector 4402 to completely surround the space around the inlet axis (co-axial with the outlet), The filter 4401 is retained by both the outlet conduit 3110 and by a rim 4403 extending distally from the deflector, an end ring 4406 of the filter engaging the deflector rim 4403.

Referring to FIGS. 48 to 50, gas trap devices 4500, 4600, and 4700 also have many parts in common with the device 3100. In the device 4500 the outlet conduit 4501 is formed by a particle mesh material engaged with a chamber 4502. There are therefore no additional parts, the outlet conduit functioning as both an outflow conduit and a particle filter. The device 4600 has a spherical particle filter 4601 surrounding the opening of the conduit 3110. The device 4700 has a particle filter 4701 having a spherical part 4702 surrounding the outlet conduit opening and a tubular insert part 4703 within the conduit.

Fluid drugs used in medical IV therapy can be formed of various chemical compositions. In some cases, where it is required to mix fluids, a chemical reaction can occur between the drugs. Similarly, in the compounding of the drugs, such as with chemotherapy, chemical reactions can occur that alter the state of the phases of the drug. Some drugs may precipitate and form solid particles, such as crystals, or carry other artefacts due to factors such as storage, half-life of the drug or agitation. The devices used to store, transport and deliver the drug into the patient may give off small particles retained from the manufacturing process that can result in small pieces of plastic, glass or other material entering the fluid, which is then infused into the patient. Whatever the cause, it is medically required to ensure that no solid particles enter the bloodstream of the patient as these can result in harm. It will be appreciated that the devices of the invention with particle filters remove such particles very effectively. Advantageously, these devices are closed system particle filter devices that also retain gases.

Fluids can have three phases—gas, liquid and solid. In IV therapy it is only the liquid drug that is of therapeutic importance and a risk of injury develops when gas (air) and/or solid particles enter the bloodstream. The invention of some embodiments reduces or eliminates the risks associated with air and particles during IV therapy, which is particularly advantageous when drugs such as chemotherapy drugs are being infused.

These devices advantageously provide a sealed chamber that isolates each of the three phases of the fluid, so that only the liquid phase is delivered beyond the chamber.

The filter membrane of any embodiment may be treated with a positive or negatively charged coating on selected areas so as to attract or repel particles. The filter may be formed of commercially available mesh material, such as those sold by Millipore or Pall, may be various size pores ranging from 0.02 mm to 0.5 mm. The filter may be formed from activated charcoal.

The illustrated position of the filters is optimal to ensure particles cannot reach the outlet port. The devices trap the gas phase within the chamber and do not allow it to exit the chamber, thereby acting as closed systems.

In one embodiment the filter device is a circular disc, the disc fixed in place. The disc can be formed as a continuous section of the device during the moulding process, or it can be fixed in place using a push fit, interference fit, snap fit, welding, bonding or any combination of these methods or any other method suitable to keep the filter in place.

The membrane may be a single membrane or a combination of membranes, such as of varying pore size, layered on top of each other or arranged in unison to prevent particles of any size from bypassing the filter. The filter may be a layered structure of mesh membranes or combined with charcoal sheet. The membrane and charcoal may have a finish applied, such as hydrophobic or hydrophilic, or be actively charged with a positive or negative coating, for the purpose of attracting or repelling liquid phases.

In one embodiment the filter/membrane is formed with a lattice of material, such as plastic, that has features enabling the disk to be attached to any internal feature of the chamber of device 100. The lattice shape and structure can be variable to ensure a full sealing of the internal chamber is achieved by the filter disk. In one embodiment the filter disk is round or elliptical in shape, having an external circumference that is equal or greater than the internal circumference of the chamber. In another embodiment the filter is square or rectangular, having external edges that are equal or greater than the internal walls of the chamber.

In one embodiment the filter membrane may be formed from only a material, such as Millipore or Pall, or any other, for the purpose of trapping particles from drugs, blood or other fluids. These particles range from 0.1 to greater than 500 microns in size. In one embodiment the filter allows fluids to flow across its entire surface and particles are trapped at any point along the inflow surface.

In a preferred embodiment the filter surface is treated with a positive or negative charge to attract particles to certain regions of the surface of the material. The particles become entwined in the material at defined locations and the untreated regions allow liquids to pass through more readily. This can have the advantage of improving flow of time-critical drugs without impedance to the flow by trapped particles.

In a system where the composite fluid entering the chamber has both air and solid phases, a diffuser device can redirect flow to accelerate degassing and flow path direction. In this embodiment the particles of solids can also be directed to certain regions of the membrane where they are less likely to impact flow rates through the membrane. The particles then settle on the treated regions of the filter, which may be to the outer edges, while liquid passes through the other regions. This has the benefit of improving drug delivery to the patient.

In one embodiment the filter is treated on its outer surface on the inflow side, to improve particle adherence on that region.

In a preferred embodiment the treated filter surface is placed on the inflow side to the chamber, with a diffuser or flow direction controlled so that the particles are guided to a preferred location across the filter face. In another embodiment the filter is placed in or about the intake end of the outflow tube. In another embodiment the filter is a wick, such as a porous rod, that is placed in or about the intake end of the outflow tube.

In a preferred embodiment the wick is a porex rod that forms the outflow tube. The outflow tube reaches a location that is adjacent to the outlet conduit opening, which is itself preferably centrally located within the chamber, or is at least located within the central 20% of any dimension through the chamber. The filter wick may advantageously have a surface treatment to attract particles. In one embodiment the filter wick is a rod of porous material. In another embodiment the filter wick is a rod of porous material with a bulbous end to increase surface area. In a preferred embodiment the filter wick is a rod of porous material with a corrugated surface to increase surface area. The benefit to this shape is that the material can capture more particles in its trough areas while the peak areas remain free of particles and promote the flow of liquid through the outlet and to the patient.

The corrugated filter device can be placed between the inlet inflow tube and the outlet outflow tube. The corrugated filter device can extend between all or part of the chamber and can be located between the inlet inflow tube and the outlet outflow tube. The corrugated filter device can be formed from solid membrane or it may have a central channel to allow liquid to pass through, the central channel forming a flow path or connecting to a flow path. The corrugated filter device can be placed about, in or over the flow path or channel. The corrugated filter device can be used in conjunction with a diverter feature to control the flow path of fluids entering the surrounding chamber.

### Bonding to the Flow Lines

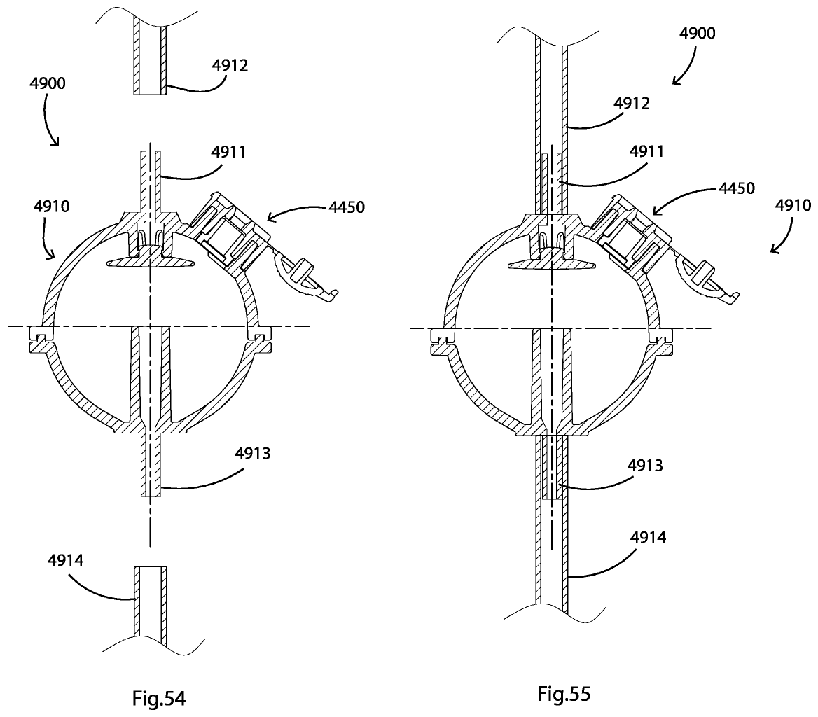

Referring to FIGS. 51, 52, and 53 a device 4800 has inlet and outlet collars 4811 and 4813 respectively which are permanently bonded to flow lines 4812 and 4814. Hence the lines and the device provide a flow assembly without need for manual connection before use. As shown in FIGS. 54 and 55, instead of collars the device (4900) may have internal tubes 4911 and 4913 for insertion into the ends of tubes 4912 and 4914 to for permanent bonding as an assembly.

Priming Port with Backcheck Valve

FIG. 56 shows various views of the priming port 4450 which is inserted into the priming port rim of a chamber of a device of any embodiment. The port 4450 has an external tubular wall 4451 and an internal tubular wall 4453 which together fit onto the chamber rim with the rim being in the cavity between the walls 4451 and 4453. A snap-fitting circumferential ridge 4452 is provided around the inside surface of the outer wall 4451 to allow easy fitting to the chamber rim. A cap 4454 is hinged by a hinge 4455 to the wall 4451 and has a central plug 4457 for fitting into a socket 4456 for full and permanent closing of the priming port after use. The cap external surface 4458 is domed, features a non-slip grip surface finish, and there are no protruding corners, and so the caregiver's gloves are unlikely to pinch at any location when closing the priming port after use. One of the drawings shows an insert 4460 which provides a membrane for performing the same function as the membrane 3308 to restrict flow of liquid out through the priming port. A latch 4459(a) is a feature of cap 4454 such that it can be closed and sealed when pressed over clip 4459(b) of wall 4451. The angled face of clip 4459(b) requires a force to applied so the mating face of latch 4459(a) can slip over the clip face. As the latch 4459(a) slips over clip 4459(b) it makes a feedback ‘click’ sound to inform the user that the latching has fully completed. Once fully latched the cap 4454 is not easily openable.

Any of the priming ports may include a one-way or backcheck valve arranged to prevent inflow of air but allow outflow of gas during priming. An example is illustrated in the figures of FIG. 63, FIG. 64 and FIG. 65. FIG. 64 shows a priming port 5000 with a main body 5001 and a cap 5002. These may be configured according to any embodiment. A backcheck valve 5010 is mounted within the port 5000. It comprises a pair of walls 5011 which converge outwardly towards each other at 5012 but are flexible enough to separate sufficiently to provide an opening 5013 under action of gas outflow during priming. This priming port is helps to prevent ambient air from flowing into the chamber and makes priming particularly convenient and simple.

Components and features of embodiments can be employed in other embodiments in a manner as would be understood by a person of ordinary skill in the art. For example, the priming port of one example can be used with a chamber of any other embodiment. The invention is not limited to the embodiments described but may be varied in construction and detail. As noted above, the priming port may be configured to be permanently or reversible closed. Also, the priming port may be closed before the gas is expelled during priming, the gas being expelled via the inlet or the outlet port. The latter is particularly possible for back-priming.

The diverter of various examples is preferably in the form of a plate, and also is preferably located at a distance between the inlet and outlet, within at least 25% of the maximum dimension of the chamber. The distance between the diverter and outflow tube is preferably at least 25% of the maximum dimension of the chamber, and, in one embodiment, within this space a particle filter is located to capture air and fluids in that 50% of the chamber dimension. In another embodiment the particle filter is located about and within the outflow tube, or forms the outflow tube itself, to capture air and particles up to 100% of the chamber volume.

The diverter is preferably formed of a flat plate of plastics material, the shape optimised to have relatively uniform thickness that can reduce shrinkage and warping of the part during the moulding process. The diverter rim features a top face on the inlet side that is 5° to 20°, to encourage flow of the fluid into the transition zone between the turbulent upper region and the laminar central region. The diverter rim features a bottom or distal face on the outlet side that is preferably 4° to 10° convex to discourage air from being trapped on this face as the device is rotated through 360°, the inclined face encouraging bubbles to slip over the surface towards the outer edge of the rim.

The diverter rim is optimally placed at the location of the inlet diffuser holes to immediately affect flow redirection, causing upward current profiles within the fluid and supporting the activity of the diffuser holes that cause the 900 redirection to accelerate degassing upon entry to the chamber. In the upright position the 10° to 20° incline on the face then motivates currents to slip into the preferred laminar flow as fluids pass down through the chamber. In the 180° inverted position the diverter rim is inclined to force fluid and air towards the outer region of the chamber and away from the centrally positioned intake end of the outflow tube. In any other orientation the diverter rim affects the flow path by encouraging the fluids and gas to rise over the outer rim, accentuating the buoyancy force for the air and the transition zone for the fluids.

Devices of the invention may be used for other applications such as delivery of fluids on very small scales such as nanoscale volumes for targeted drug delivery. In such cases the above dimension ranges may have lower values as low as 0.01 mm. Other applications may be in manufacturing or fuel systems.

Methods of use may advantageously attach or include an air trapping device of the invention in conjunction with a particle trapping device on an infusion tubing set to remove air or particles from an infusion fluid and to maintain a closed system during IV infusion proximal or distal to an IV pump or active device.

The method of reversing the flow in either forwards or backwards direction through an air trapping device in conjunction with a particle trapping device, either in series or as a combination product, helps to aspirate air into a reservoir and maintain a closed system during IV infusion.

1-70. (canceled)

71. A gas trap device for medical supply of liquids to a human or animal body,
the device comprising:
a chamber,
an inlet port,
a diffuser in the inlet port, the diffuser comprising openings for lateral flow into the chamber of fluid relative to an inlet port axis,
an outlet port spaced apart distally from the inlet port and being linked with a conduit within the chamber and the conduit having an opening within the chamber,
a flow diverter mounted distally of the inlet port and proximally of the conduit, and
a single-use priming port, configured to allow venting of the chamber during priming and to be permanently sealed upon completion of priming.

72. The gas trap device of claim 71, wherein the diverter is configured for flow of inlet fluid to change direction through at least 80° from an inlet port axis and then to flow radially through the diffuser openings and the openings have an area of less than half of an area around the axis.

73. The gas trap device of claim 71, wherein the diffuser openings have an area which is in the range of 20% to 40% of an area around the inlet axis.

74. The gas trap device of claim 71, wherein there are in the range of two to four diffuser openings.

75. The gas trap device of claim 71, wherein the diverter is located within the chamber in a region less than 40% from a proximal end of the chamber, and preferably less than 25% from the proximal end of the chamber.

76. The gas trap device of claim 71, wherein the diverter forms an end surface of the inlet port, forcing inlet fluid to flow through the diffuser openings.

77. The gas trap device of claim 71, wherein the priming port comprises a body forming a vent opening and a cap.

78. The gas trap device of claim 77, wherein the cap and the vent opening have inter-engaging features, which are engageable by pushing the cap into the vent opening.

79. The gas trap device of claim 77, wherein the cap and the vent opening comprise inter-engaging ridge and groove.

80. The gas trap device of claim 77, wherein the cap has a curved exposed surface.

81. A gas trap device for medical supply of liquids to a human or animal body,
the device comprising:
a spherical-shaped chamber having a proximal inlet side and a distal outlet side,
an inlet port,
a diffuser in the inlet port, the diffuser comprising openings for lateral flow into the chamber of fluid relative to an inlet port axis,
an outlet port spaced apart distally from the inlet port and being linked with a conduit within the chamber and the conduit having an opening within the chamber,
a flow diverter mounted distally of the inlet port and proximally of the conduit, and
a single-use priming port, configured to allow venting of the chamber during priming, disposed at the proximal inlet side of the chamber.

82. The gas trap device of claim 81, wherein the priming port is disposed adjacent the inlet port.

83. The gas trap device of claim 81, wherein the priming port is proximal to the diffuser.

84. The gas trap device of claim 81, wherein a priming port connector extends parallel to the inlet port.

85. The gas trap device of claim 81, wherein the priming port comprises a body forming a vent opening and a cap.

86. A method of use of a gas trap device comprising a chamber, an inlet port, an outlet port spaced apart distally from the inlet port, a flow diverter within the chamber at a distal end of the inlet port, and a priming port, the method comprising:
allowing flow of a priming fluid into the chamber to cause the chamber to fill with the priming fluid and expel gas from the chamber, via the priming port,
closing the priming port after expelling the gas, and
allowing a fluid to flow through the gas trap device.

87. The method of claim 86, wherein the priming fluid is saline.

88. The method of claim 86, wherein either the priming port or the outlet port is closed and the priming fluid is allowed to flow through the inlet port until it fills the chamber, upon which the priming port is sealed and flow is prevented through the inlet until the outlet is opened.

89. The method of claim 86, wherein the inlet port is closed, the outlet port is connected to a connector for a primary line, a line distally of the connector is closed, a flow is allowed through the primary line so that it is diverted via the connector back upwardly into the device until it fills the device chamber, the priming port is sealed, the line distally of the connector is opened, and the inlet port is connected to a secondary line.

90. The method of claim 86, wherein entrained air from the device attached to an IV line is expelled during priming.
